(19) United States
(12) Patent Application Publication
(10) Pub. No.: US 2021/0324067 A9
(43) Pub. Date: Oct. 21, 2021

(54) COMBINATION THERAPY FOR CARDIOVASCULAR DISEASES

(71) Applicant: THE BRIGHAM AND WOMEN'S HOSPITAL, INC., Boston, MA (US)

(72) Inventor: Paul M. Ridker, Chestnut Hill, MA (US)

(73) Assignee: The Brigham and Women's Hospital, Inc., Boston, MA (US)

(21) Appl. No.: 16/978,660

(22) PCT Filed: Mar. 8, 2019
(86) PCT No.: PCT/US2019/021361

§ 371 Date: Sep. 4, 2020

Related U.S. Application Data
(60) Provisional application No. 62/733,960, filed on Sep. 20, 2018.
(60) Provisional application No. 62/640,918, filed on Mar. 9, 2018.

(51) Int. Cl. C07K 16/24; A61P 9/00; A61K 31/366; C12N 15/113; A61K 31/22; A61K 31/405; A61K 31/4418; A61K 31/47; A61K 31/505; C07K 16/40

(52) U.S. Cl. CPC C07K 16/245; A61P 9/00; A61K 31/366; C12N 15/1136; A61K 31/22; C07K 16/40; A61K 31/4418; A61K 31/47; A61K 31/505; C12N 15/1138; A61K 31/405

(57) ABSTRACT

Provided herein are methods of treating or reducing the risk of a cardiovascular disease using a lipid lowering agent (e.g., statin and/or PCSK9 inhibitor) and an anti-inflammatory agent (e.g., a pro-inflammatory cytokine inhibitor). Further provided herein are methods of predicting the recurrence rate of a subject who has received or is undergoing therapy for a cardiovascular disease with a lipid lowering agent on the basis of the C-reactive protein (CRP) level in the subject. In some embodiments, the recurrence rate can be reduced using an anti-inflammatory agent.

### RELATED APPLICATION

This application claims the benefit under 35 U.S.C. § 119(e) of U.S. Provisional Patent Application No. 62/640,918, filed Mar. 9, 2018, and entitled “COMBINATION THERAPY FOR CARDIOVASCULAR DISEASES,” and claims the benefit under 35 U.S.C. § 119(e) of U.S. Provisional Patent Application No. 62/733,960, filed Sep. 20, 2018, and entitled “COMBINATION THERAPY FOR CARDIOVASCULAR DISEASES,” the entire contents of each of which are incorporated herein by reference.

### BACKGROUND

Lipid reduction is a mainstay in the treatment of atherosclerotic cardiovascular disease. Some patients continue to have cardiovascular disease despite being on lipid-lowering therapy. There is a need to develop new treatments.

### SUMMARY

The present disclosure, in some aspects, is based on the surprising finding that residual inflammatory risk exists in patients that have been undergoing aggressive lipid-lowering therapy, and that the high sensitivity C-reactive protein (hsCRP) level (a marker of inflammation) in these patients correlates with the likelihood of recurrence of the cardiovascular diseases, and/or mortality rate. Provided herein are methods of treating cardiovascular diseases using a lipid lowering agent and an anti-inflammatory agent.

Some aspects of the present disclosure provide methods of treating a cardiovascular disease, the method comprising administering to a subject in need thereof a therapeutically effective amount of a lipid lowering agent and an anti-inflammatory agent.

In some embodiments, the anti-inflammatory agent is a proinflammatory cytokine inhibitor. In some embodiments, the anti-inflammatory agent comprises an IL-1 inhibitor, an IL-1 receptor (IL-1R) inhibitor, an IL-6 inhibitor, an IL-6 receptor (IL-6R) inhibitor, a NLRP3 inhibitor, a TNF inhibitor, an IL-8 inhibitor, an IL-18 inhibitor, an inhibitor of natural killer cells, or combinations thereof. In some embodiments, the anti-inflammatory agent is a nucleic acid, an aptamer, an antibody or antibody fragment, an inhibitory peptide, or a small molecule.

In some embodiments, the anti-inflammatory agent comprises an IL-1 inhibitor. In some embodiments, the IL-1 inhibitor is an IL-1α inhibitor. In some embodiments, the IL-1αinhibitor is an anti-sense oligonucleotide against IL-1α, MABp1, or sIL-1RI. In some embodiments, the IL-1 inhibitor is an IL-1β inhibitor. In some embodiments, the IL-1β inhibitor is an anti-sense oligonucleotides against IL-1β, canakinumab, diacerein, gevokizumab, LY2189102, CYT013, sIL-1RII, VX-740, or VX-765. In some embodiments, the IL-1 inhibitor is suramin sodium, methotrexate-methyl-d3, methotrexate-methyl-d3 dimethyl ester, or diacerein.

In some embodiments, the anti-inflammatory agent comprises an IL-1R inhibitor. In some embodiments, the IL-1R inhibitor is an IL-1R antagonist. In some embodiments, the IL-1R inhibitor is an anti-sense oligonucleotide against IL-1R, anakinra, Rilonacept, MEDI-8968, sIL-1RI, EBI-005, interleukin-1 receptor antagonist (IL-RA), or AMG108.

In some embodiments, the anti-inflammatory agent comprises an IL-6 inhibitor. In some embodiments, the IL-6 inhibitor is an anti-sense oligonucleotide against IL-6, siltuximab, sirukumab, clazakizumab, olokizumab, elsilimomab, IG61, BE-8, CNT0328 PGE1 and its derivatives, PGI2 and its derivatives, or cyclophosphamide.

In some embodiments, the anti-inflammatory agent comprises an IL-6R inhibitor. In some embodiments, the IL-6R inhibitor is an IL-6R antagonist. In some embodiments, the IL-6R inhibitor is an anti-sense oligonucleotide against IL-6R, tocilizumab, sarilumab, PM1, AUK12-20, AUK64-7, AUK146-15, MRA, or AB-227-NA.

In some embodiments, the anti-inflammatory agent comprises a NLRP3 inhibitor. In some embodiments, the NLPR3 inhibitor is an anti-sense oligonucleotide against NLPR3, colchicine, MCC950, CY-09, ketone metabolite beta-hydroxubutyrate (BHB), a type I interferon, resveratrol, arglabin, CB2R, Glybenclamide, Isoliquiritigenin, Z-VAD-FMK, or microRNA-223.

In some embodiments, the anti-inflammatory agent comprises a TNF inhibitor. In some embodiments, the TNF inhibitor is an anti-sense oligonucleotide against TNF, infliximab, adalimumab, certolizumab pegol, golimumab, etanercept (Enbrel), thalidomide, lenalidomide, pomalidomide, a xanthine derivative, bupropion, 5-HT2A agonist or a hallucinogen.

In some embodiments, the anti-inflammatory agent comprises an IL-8 inhibitor. In some embodiments, the IL-8 inhibitor is an anti-sense oligonucleotides against IL8, HuMab-10F8, Reparixin, Curcumin, Antileukinate, Macrolide, or a trifluoroacetate salt.

In some embodiments, the anti-inflammatory agent comprises an IL-18 inhibitor. In some embodiments, the IL-18 inhibitor is selected from the group consisting of: anti-sense oligonucleotides against IL-18, IL-18 binding protein, IL-18 antibody, NSC201631, NSC61610, and NSC80734.

In some embodiments, the anti-inflammatory agent comprises an inhibitor of natural killer cells. In some embodiments, the inhibitor of natural killer cells is an antibody targeting natural killer cells.

In some embodiments, the anti-inflammatory agent comprises methotrexate. In some embodiments, the anti-inflammatory agent comprises arhalofenate.

In some embodiments, the lipid lowering agent comprises a proprotein convertase subtilisin/kexin type 9 (PCSK9) inhibitor. In some embodiments, the PCSK9 inhibitor is a natural PCSK9 inhibitor, an anti-PCSK9 antibody, an antisense nucleic acid, a peptide inhibitor, a PCSK9 vaccine, or a small molecule inhibitor. In some embodiments, the natural PCSK9 inhibitor is berberine, annexin A2, or adnectin. In some embodiments, the small molecule inhibitor is PF-06446846, anacetrapib, or K-312. In some embodiments, the PCSK9 antibody is alirocumab, evolocumab, 1D05-IgG2, RG-7652, LY3015014, or bococizumab. In some embodiments, the antisense nucleic acid is an RNAi molecule. In some embodiments, the RNAi molecule is inclisiran or ALN-PCS. In some embodiments, the peptide inhibitor is a peptide that mimics an EGFa domain of low-density lipoprotein receptor (LDL-R). In some embodiments, the PCSK9 vaccine comprises an antigenic PCSK9 peptide.

In some embodiments, the lipid lowering agent comprises a HMG-CoA reductase inhibitor. In some embodiments, the HMG-CoA reductase inhibitor is a statin. In some embodiments, the statin is simvastatin, lovastatin, pravastatin, fluvastatin, atorvastatin, cerivastatin, rosuvastatin, or pitivastatin.

In some embodiments, the lipid lowering agent a fibric acid derivative (fibrate), a bile acid sequestrant or resin, a nicotinic acid agent, a cholesterol absorption inhibitor, acyl-coenzyme A, a cholesterol acyl transferase (ACAT) inhibitor, a cholesteryl ester transfer protein (CETP) inhibitor, an LDL receptor antagonist, a farnesoid X receptor (FXR) antagonist, a sterol regulatory binding protein cleavage activating protein (SCAP) activator, a microsomal triglyceride transfer protein (MTP) inhibitor, a squalene synthase inhibitor, or a peroxisome proliferation activated receptor (PPAR) agonist.

In some embodiments, the lipid lowering agent and the anti-inflammatory agent are administered together. In some embodiments, the lipid lowering agent and the anti-inflammatory agent are administered separately. In some embodiments, the lipid lowering agent and/or the anti-inflammatory agent is administered intravenously, intramuscularly, subcutaneously, or orally.

In some embodiments, the level or activity of a proinflammatory cytokine in the subject is reduced. In some embodiments, the level or activity of C-reactive protein (CRP) in the subject is reduced. In some embodiments, the level or activity of non-high-density lipoprotein (HDL)-cholesterol in the subject is reduced. In some embodiments, the level or activity of LDL-cholesterol in the subject is reduced. In some embodiments, the level or activity of total cholesterol in the subject is reduced. In some embodiments, the level or activity of apolipoprotein B (ApoB) in the subject is reduced. In some embodiments, the level or activity of triglycerides in the subject is reduced. In some embodiments, the ratio of total cholesterol to HDL-cholesterol in the subject is reduced. In some embodiments, the occurrence of non-fatal myocardial infarction is reduced. In some embodiments, the occurrence of non-fatal stroke is reduced. In some embodiments, the rate of cardiovascular mortality is reduced.

In some embodiments, the cardiovascular disease is myocardial infarction, stroke, acute coronary syndrome, myocardial ischemia, chronic stable angina pectoris, unstable angina pectoris, cardiovascular death, coronary re-stenosis, coronary stent re-stenosis, coronary stent re-thrombosis, revascularization, angioplasty, transient ischemic attack, pulmonary embolism, vascular occlusion, or venous thrombosis.

Other aspects of the present disclosure provide methods of reducing a recurrence rate of a cardiovascular disease in a subject who has received or is undergoing therapy with a lipid lowering agent, the method comprising administering to the subject an effective amount of an anti-inflammatory agent.

Other aspects of the present disclosure provide methods of predicting a recurrence rate of a cardiovascular disease in a subject who has received or is undergoing therapy with the lipid lowering agent, the method comprising measuring a level of C-reactive protein (CRP) in the subject and determining that the subject is likely to have recurrence of the cardiovascular disease if the CRP level is above a pre-determined value. In some embodiments, the pre-determined value is 3 mg/L. In some embodiments, the pre-determined value is 2 mg/L. In some embodiments, the pre-determined value is 1 mg/L.

Further provided herein are methods of treating a cardiovascular disease, the method comprising administering to a subject in need thereof a therapeutically effective amount of a bispecific antibody comprising a first antigen-binding domain that binds an proinflammatory cytokine and a second antigen-binding domain that binds a proprotein convertase subtilisin/kexin type 9 (PCSK9). In some embodiments, the proinflammatory cytokine is selected from IL-1, IL-1 receptor (IL-1R), IL-6, IL-6 receptor (IL-6R), NLRP3, TNF, IL-8, or IL-18.

In some embodiments, the first antigen-binding domain binds to IL-1. In some embodiments, the first antigen-binding domain binds to IL-1α. In some embodiments, the first antigen-binding domain is derived from MABp1. In some embodiments, the first antigen-binding domain binds to IL-1β. In some embodiments, the first antigen-binding domain is derived from canakinumab, diacerein, gevokizumab, or LY2189102. In some embodiments, the first antigen-binding domain binds to IL-1R. In some embodiments, the first antigen-binding domain is derived from MEDI-8968 or AMG108. In some embodiments, the first antigen-binding domain binds to IL-6. In some embodiments, the first antigen-binding domain is derived from siltuximab, sirukumab, clazakizumab, olokizumab, or elsilimomab. In some embodiments, the first antigen-binding domain binds to IL-6R. In some embodiments, the first antigen-binding domain is derived from tocilizumab, sarilumab, PM1, AUK12-20, AUK64-7, AUK146-15, or AB-227-NA. In some embodiments, the first antigen-binding domain binds to NLRP3. In some embodiments, the first antigen-binding domain is derived from a NLRP3 antibody. In some embodiments, the first antigen-binding domain binds to TNF. In some embodiments, the first antigen-binding domain is derived from infliximab, adalimumab, certolizumab pegol, golimumab, or etanercept (Enbrel). In some embodiments, the first antigen-binding domain binds to IL-8. In some embodiments, the first antigen-binding domain is derived from HuMab-10F8. In some embodiments, the first antigen-binding domain binds to IL-18. In some embodiments, the first antigen-binding domain is derived from a IL-18 antibody.

In some embodiments, the second antigen-binding domain is derived from alirocumab, evolocumab, 1D05-IgG2, RG-7652, LY3015014, or bococizumab.

In some embodiments, the bispecific antibody comprises a common Fc region. In some embodiments, the bispecific antibody is a monoclonal bispecific antibody.

In some embodiments, the method further comprises administering to the subject a therapeutically effective amount of a HMG-CoA reductase inhibitor. In some embodiments, the HMG-CoA reductase inhibitor is a statin. In some embodiments, the statin is simvastatin, lovastatin, pravastatin, fluvastatin, atorvastatin, cerivastatin, rosuvastatin, or pitivastatin.

In some embodiments, the bispecific antibody is administered intravenously, intramuscularly, subcutaneously, or orally. In some embodiments, the level or activity of a proinflammatory cytokine in the subject is reduced. In some embodiments, the level or activity of C-reactive protein (CRP) in the subject is reduced. In some embodiments, the level or activity of non-high-density lipoprotein (HDL)-cholesterol in the subject is reduced. In some embodiments, the level or activity of LDL-cholesterol in the subject is reduced. In some embodiments, the level or activity of total cholesterol in the subject is reduced. In some embodiments, the level or activity of apolipoprotein B (ApoB) in the subject is reduced. In some embodiments, the level or activity of triglycerides in the subject is reduced. In some embodiments, the ratio of total cholesterol to HDL-cholesterol in the subject is reduced. In some embodiments, the occurrence of non-fatal myocardial infarction is reduced. In some embodiments, the occurrence of non-fatal stroke is reduced. In some embodiments, the rate of cardiovascular mortality is reduced.

Further provided herein are methods of treating a cardiovascular disease, the method comprising administering to a subject in need thereof a therapeutically effective amount of a bispecific antibody comprising a first antigen-binding domain that binds IL-1 and a second antigen-binding domain that binds a proprotein convertase subtilisin/kexin type 9 (PCSK9).

In some embodiments, the first antigen-binding domain binds to IL-1α. In some embodiments, the first antigen-binding domain is derived from MABp1. In some embodiments, the first antigen-binding domain binds to IL-1β. In some embodiments, the first antigen-binding domain is derived from canakinumab, diacerein, gevokizumab, or LY2189102. In some embodiments, the second antigen-binding domain is derived from alirocumab, evolocumab, 1D05-IgG2, RG-7652, LY3015014, or bococizumab.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIGS. 1A-1D show the mean percentage change in lipid levels from baseline to 14 weeks according to hsCRP_{OT}. Median on-treatment lipid values (FIG. 1A, Total cholesterol; FIG. 1B, LDL cholesterol; FIG. 1C, HDL cholesterol; and FIG. 1D, TC:HDL-C ratio) in each hsCRP_{OT} group are shown to the right of each plot. HDL-C indicates high-density lipoprotein cholesterol; hsCRP_{OT}, on-treatment levels of high-sensitivity C-reactive protein; LDL-C, low-density lipoproteincholesterol; and TC, total cholesterol.

FIG. 2 shows the relationship between hsCRP_{OT} on a continuous scale and the adjusted event rate for the trial primary end point (myocardial infarction, stroke, unstable angina requiring urgent coronary revascularization, and cardiovascular death). Model adjusts for age, sex, current smoking, diabetes mellitus, hypertension, body mass index, statin intensity at enrollment (moderate or high), and on-treatment levels of low-density lipoprotein cholesterol. Dots represent individual hsCRP_{OT} values. hsCRP_{OT} indicates on-treatment levels of high-sensitivity C-reactive protein.

FIGS. 3A-3B show the risk association of hsCRP_{OT} and LDL-C_{OT} with incident cardiovascular events according to categories of each biomarker. Adjusted for age, sex, current smoking, diabetes mellitus, hypertension, body mass index, statin intensity at enrollment (moderate or high), and hsCRP_{OT} and LDL-C_{OT} as appropriate. FIG. 3A shows models for hsCRP_{OT}. FIG. 3B shows models for LDL-C_{OT}. CI indicates confidence interval; hsCRP_{OT}, on-treatment levels of high-sensitivity C-reactiveprotein; LDL-C_{OT}, on-treatment levels of low-density lipoprotein cholesterol; and Ref, reference.

### DETAILED DESCRIPTION OF CERTAIN EMBODIMENTS

Despite aggressive lipid lowering therapies, patients continue to have cardiovascular disease. We found that among primary as well as secondary prevention patients already on aggressive LDL-C lowering therapy with both statins and a PCSK9 inhibitor there is still clear evidence of residual inflammatory risk on the basis of on-treatment hsCRP levels. Prior to the present disclosure, it was still uncertain whether residual inflammatory risk persists after extremely aggressive reduction in LDL-C.

Some aspects of the present disclosure is based, at least in part, on the surprising finding that in a population of 9,738 high-risk patients aggressively treated with lipid lowering agent (e.g., concomitantly treated with statins and PSCK9 inhibition), a large percentage of patients, despite exceptionally aggressive reduction of lipids, are still at a continuous gradient in risk for future cardiovascular diseases. Such patients exhibit a higher than normal on-treatment hsCRP level. Compared to those without evidence of subclinical inflammation, those with on-treatment hsCRP>3 mg/L had a 62% increase in risk of future vascular events. Elevated hsCRP was significantly associated with increased rates of myocardial infarction, cardiovascular death, and/or all-cause mortality. We believe that inflammation risk persists despite aggressive maximal LDL-C lowering, and that inflammation reduction provides additional benefit for cardiovascular disease reduction.

Accordingly, some aspects of the present disclosure provide methods of treating a cardiovascular disease, the method comprising administering to a subject in need thereof a therapeutically effective amount of a lipid lowering agent and an anti-inflammatory agent.

An “anti-inflammatory agent” refers to an agent that reduces inflammation or inflammatory response. In some embodiments, the anti-inflammatory agent is a proinflammatory cytokine inhibitor. A “proinflammatory cytokine inhibitor” refers to an agent that inhibits the inflammatory signaling pathway induced by proinflammatory cytokines. A proinflammatory cytokine inhibitor may inhibit the level or activity of any protein or nucleic acid involved in the inflammatory signaling pathway. For example, in some embodiments, the proinflammatory cytokine inhibitor inhibits the level of proinflammatory cytokines (e.g., IL-1 such as IL-1α and Il-1β, IL-6, IL-8, and IL-18). In some embodiments, the proinflammatory cytokine inhibitor inhibits the activity of proinflammatory cytokines, e.g., by inhibiting the level or activity of cytokine receptors (e.g., IL-1R and IL-6R).

In some embodiments, the proinflammatory cytokine inhibitor inhibits the inflammasome. The inflammasome is a multiprotein oligomer expressed in myeloid cells and is a component of the innate immune system. The exact composition of an inflammasome depends on the activator which initiates inflammasome assembly, e.g. dsRNA will trigger one inflammasome composition whereas asbestos will assemble a different variant. The inflammasome promotes the maturation of the inflammatory cytokines Interleukin 1β (IL-1β) and Interleukin 18 (IL-18). In some embodiments, the inflammasome consists of caspase 1, PYCARD or ASC, NALP and sometimes caspase 5 (also known as caspase 11 or ICH-3). In some embodiments, the inflammasome contains nod-like receptor protein 3 (NLRP3).

In some embodiments, the anti-inflammatory agent is a nucleic acid, an aptamer, an antibody or antibody fragment, an inhibitory peptide, or a small molecule. In some embodiments, the anti-inflammatory agent is an inhibitory nucleic acid, such as an antisense nucleic acid designed to target a proinflammatory cytokine gene.

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In some embodiments, the antisense nucleic acid is a RNAi molecule. A RNAi molecule is an antisense molecule that inhibits expression of a proinflammatory cytokine signaling component. The nucleic acid sequences of proinflammatory cytokines are known in the art. The inhibitory nucleic acids may be designed using routine methods in the art.

An inhibitory nucleic acid (e.g., an anti-sense oligonucleotide) against a proinflammatory cytokine gene typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In some embodiments, vector-based RNAi modalities (e.g., shRNA or shRNA-mir expression constructs) are used to reduce expression of a gene (e.g., a target nucleic acid such as a proinflammatory cytokine nucleic acid) in a cell. In some embodiments, the inhibitory nucleic acid comprises an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some embodiments, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based molecules could be employed to inhibit expression of a gene (e.g., a proinflammatory cytokine gene) in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to S nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of a gene (e.g., a CSC-associated gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins.

Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

In some embodiments, inhibitory nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33).

In some embodiments, the inhibitory nucleic acid of the present disclosure is 100% identical to the nucleic acid target. In other embodiments it is at least 99%, 95%, 90%, 85%, 80%, 75%, 70%, or 50% identical to the nucleic acid target. The term “percent identical” refers to sequence identity between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ-FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

An inhibitory nucleic acid useful in the present disclosure will generally be designed to have partial or complete complementarity with one or more target genes (i.e., complementarity with one or more transcripts of a proinflammatory cytokine gene). The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition, which may be greater than 10%, 20%, 30%, 40%, 50%, 60%, 70%, 80%, 90%, 95% or 99% as compared to a cell not treated according to the present disclosure. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

“Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody. An antibody may be a chimeric antibody or a humanized antibody.

An “antibody fragment” for use in accordance with the present disclosure contains the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

“Inhibitory peptides” refers to peptides that specifically binds to a target molecule. In some embodiments, binding of an inhibitory peptide to a target molecule inhibits the biological activity of the target molecule. For example, if the target molecule functions in a signaling pathway, binding of the inhibitory peptide may inhibit the signaling pathway. One skilled in the art is familiar with inhibitory peptides or methods of developing inhibitory peptides to their target molecule of choice. For example, peptides derived from the receptor binding portion of proinflammatory cytokines may competitively bind to the receptor, preventing binding of the cytokine and inhibiting downstream signaling. An inhibitory peptides may also be synthetic (i.e., synthetic peptides). One skilled in the art is familiar with methods of designing and synthesizing inhibitory peptides.

An “aptamer” refers to an oligonucleotide or a peptide molecule that binds to a specific target molecule. Aptamers are usually created by selecting them from a large random sequence pool.

A “small molecule,” as used herein, refers to a molecule of low molecular weight (e.g., <900 daltons) organic or inorganic compound that may function in regulating a biological process. whether naturally-occurring or artificially created (e.g., via chemical synthesis) that has a relatively low molecular weight. Typically, an organic compound contains carbon. An organic compound may contain multiple carbon-carbon bonds, stereocenters, and other functional groups (e.g., amines, hydroxyl, carbonyls, or heterocyclic rings). In some embodiments, small molecules are monomeric organic compounds that have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body. In certain embodiments, the organic molecule is known to bind and/or cleave a nucleic acid. In some embodiments, the organic compound is an enediyne. Non-limiting examples of a small molecule include lipids, monosaccharides, second messengers, other natural products and metabolites, as well as drugs and other xenobiotics.

A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relay signals received at receptors on the cell surface (e.g., from protein hormones, growth factors, etc.) to target molecules in the cytosol and/or nucleus. Non-limiting examples of second messenger molecules include cyclic AMP, cyclic GMP, inositol trisphosphate, diacylglycerol, and calcium. A “metabolite” is an molecule that forms as an intermediate produce of metabolism. Non-limiting examples of a metabolite include ethanol, glutamic acid, aspartic acid, 5′ guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs, antibiotics, carcinogens, environmental pollutants, food additives, hydrocarbons, and pesticides.

In some embodiments, the anti-inflammatory agent is selected from the group consisting of: IL-1 inhibitors, IL-1 receptor (IL-1R) inhibitors, IL-6 inhibitors, IL-6 receptor (IL-6R) inhibitors, NLRP3 inhibitors, TNF inhibitors, IL-8 inhibitors, IL-18 inhibitors, or inhibitors of natural killer cells. Combinations of different anti-inflammatory agents described herein are contemplated. In some embodiments, the anti-inflammatory agent comprises inhibitors to one or more (e.g., 1, 2, 3, 4, 5, 6, 7, 8, 9, 10, or more) proinflammatory cytokines. Each inhibitor to one proinflammatory cytokine may be a combination of different types of inhibitors, e.g., inhibitory nucleic acids, inhibitory peptides, antibodies, or small molecules.

In some embodiments, the anti-inflammatory agent may be: a combination of an IL-1 inhibitor and an IL-1R inhibitor; a combination of an IL-1 inhibitor and an IL-6 inhibitor; a combination of an IL-1 inhibitor and an IL-6R inhibitor; a combination of an IL-1 inhibitor and a NLRP3 inhibitor; a combination of an IL-1 inhibitor and a TNF inhibitor; a combination of an IL-1 inhibitor and an IL-8 inhibitor; a combination of an IL-1 inhibitor and an IL-18 inhibitor; a combination of an IL-1R inhibitor and an IL-6 inhibitor; a combination of an IL-1R inhibitor and an IL-6R inhibitor; a combination of an IL-1R inhibitor and a NLRP3 inhibitor; a combination of an IL-1R inhibitor and a TNF inhibitor; a combination of an IL-1R inhibitor and an IL-8 inhibitor; a combination of an IL-1R inhibitor and an inhibitor of natural killer cells, a combination of an IL-1R inhibitor and an IL-18 inhibitor; a combination of an IL-6 inhibitor and an IL-6R inhibitor; a combination of an IL-6 inhibitor and a NLRP3 inhibitor; a combination of an IL-6 inhibitor and a TNF inhibitor; a combination of an IL-6 inhibitor and an IL-8 inhibitor; a combination of an IL-6 inhibitor and an IL-18 inhibitor; a combination of an IL-6 inhibitor and an inhibitor of natural killer cells; a combination of an IL-6R inhibitor and a NLRP3 inhibitor; a combination of an IL-6R inhibitor and a TNF inhibitor; a combination of an IL-6R inhibitor and an IL-8 inhibitor; a combination of an IL-6R inhibitor and an IL-18 inhibitor; a combination of an IL-6R inhibitor and an inhibitor of natural killer cells; a combination of a NLRP3 inhibitor and a TNF inhibitor; a combination of a NLRP3 inhibitor and an IL-8 inhibitor; a combination of a NLRP3 inhibitor and an IL-18 inhibitor; a combination of a NLRP3 inhibitor and an inhibitor of natural killer cells; a combination of an IL-8 inhibitor and an IL-18 inhibitor; a combination of an IL-8 inhibitor and an inhibitor of natural killer cells; a combination of an IL-1 inhibitor, an IL-1R inhibitor, and an IL-6 inhibitor; a combination of an IL-1 inhibitor, an IL-1R inhibitor, and an IL-6R inhibitor; a combination of an IL-1 inhibitor, an IL-1R inhibitor, and a NLRP3 inhibitor; a combination of an IL-1 inhibitor, an IL-1R inhibitor, and a TNF inhibitor; a combination of an IL-1 inhibitor, an IL-1R inhibitor, and an IL-8 inhibitor; a combination of an IL-1 inhibitor, an IL-1R inhibitor, and an IL-18 inhibitor; a combination of an IL-1 inhibitor, an IL-1R inhibitor, and an inhibitor of natural killer cells; a combination of an IL-1 inhibitor, an IL-6 inhibitor, and an IL-6R inhibitor; a combination of an IL-1 inhibitor, an IL-6 inhibitor, and a NLRP3 inhibitor; a combination of an IL-1 inhibitor, an IL-6 inhibitor, and a TNF inhibitor; a combination of an IL-1 inhibitor, an IL-6 inhibitor, and an IL-8 inhibitor; a combination of an IL-1 inhibitor, an IL-6 inhibitor, and an IL-18 inhibitor; a combination of an IL-1 inhibitor, an IL-6R inhibitor, and a NLRP3 inhibitor; a combination of an IL-1 inhibitor, an IL-6R inhibitor, and a TNF inhibitor; a combination of an IL-1 inhibitor, an IL-6R inhibitor, and an IL-8 inhibitor; a combination of an IL-1 inhibitor, an IL-6R inhibitor, and an IL-18 inhibitor; a combination of an IL-1 inhibitor, an IL-6R inhibitor, and an inhibitor of natural killer cells; a combination of an IL-1 inhibitor, a NLRP3 inhibitor, and a TNF inhibitor; a combination of an IL-1 inhibitor, a NLRP3 inhibitor, and an IL-8 inhibitor; a combination of an IL-1 inhibitor, a NLRP3 inhibitor, and an inhibitor of natural killer cells; a combination of an IL-1 inhibitor, a NLRP3 inhibitor, and an IL-18 inhibitor; a combination of an IL-1 inhibitor, a TNF inhibitor, and an IL-8 inhibitor; a combination of an IL-1 inhibitor, a TNF inhibitor, and an IL-18 inhibitor; a combination of an IL-1 inhibitor, a TNF inhibitor, and an inhibitor of natural killer cells; a combination of an IL-1 inhibitor, an IL-8 inhibitor, and an IL18 inhibitor; a combination of an IL-1 inhibitor, an IL-8 inhibitor, and an inhibitor of natural killer cells; a combination of an IL-1 inhibitor, a NLRP3 inhibitor, and a TNF inhibitor; a combination of an IL-1R inhibitor, an IL-6 inhibitor, and an IL-6R inhibitor; a combination of an IL-1R inhibitor, an IL-6 inhibitor, and a NLRP3 inhibitor; a combination of an IL-1R inhibitor, an IL-6 inhibitor, and a TNF inhibitor; a combination of an IL-1R inhibitor, an IL-6 inhibitor, and an IL-8 inhibitor; a combination of an IL-1R inhibitor, an IL-6 inhibitor, and an IL-18 inhibitor; a combination of an IL-1R inhibitor, an IL-6 inhibitor, and an inhibitor of natural killer cells; a combination of an IL-1R inhibitor, an IL-6R inhibitor, and a NLRP3 inhibitor; a combination of an IL-1R inhibitor, an IL-6R inhibitor, and a TNF inhibitor; a combination of an IL-1R inhibitor, an IL-6R inhibitor, and an IL-8 inhibitor; a combination of an IL-1R inhibitor, an IL-6R inhibitor, and an IL-18 inhibitor; a combination of an IL-1R inhibitor, an IL-6R inhibitor, and an inhibitor of natural killer cells; a combination of an IL-1R inhibitor, a NLRP3 inhibitor, and a TNF inhibitor; a combination of an IL-1R inhibitor, a NLRP3 inhibitor, and an IL-8 inhibitor; IL-1R inhibitor, a NLRP3 inhibitor, and an inhibitor of natural killer cells; a combination of an IL-1R inhibitor, a NLRP3 inhibitor, and an IL-18 inhibitor; a combination of an IL-1R inhibitor, a TNF inhibitor, and an IL-8 inhibitor; a combination of an IL-1R inhibitor, a TNF inhibitor, and an Il-18 inhibitor; a combination of an IL-1R inhibitor, a TNF inhibitor, and an inhibitor of natural killer cells; a combination of an IL-1R inhibitor, an IL-8 inhibitor, and an IL18 inhibitor; a combination of an IL-1R inhibitor, an IL-8 inhibitor, and an inhibitor of natural killer cells; a combination of an IL-6 inhibitor, a NLRP3 inhibitor, and a TNF inhibitor; a combination of an IL-6 inhibitor, an IL-6R inhibitor, and a NLRP3 inhibitor; a combination of an IL-6 inhibitor, an IL-6R inhibitor, and a TNF inhibitor; a combination of an IL-6 inhibitor, an IL-6R inhibitor, and an IL-8 inhibitor; a combination of an IL-6 inhibitor, an IL-6R inhibitor, and an IL-18 inhibitor; a combination of an IL-6 inhibitor, an IL-6R inhibitor, and an inhibitor of naturally killer cells; a combination of an IL-6 inhibitor, a NLRP3 inhibitor, and a TNF inhibitor; a combination of an IL-6 inhibitor, a NLRP3 inhibitor, and an IL-8 inhibitor; a combination of an IL-6 inhibitor, a NLRP3 inhibitor, and an IL-18 inhibitor; a combination of an IL-6 inhibitor, a NLRP3 inhibitor, and an inhibitor of natural killer cells; a combination of an IL-6 inhibitor, a TNF inhibitor, and an IL-8 inhibitor; a combination of an IL-6 inhibitor, a TNF inhibitor, and an IL-18 inhibitor; a combination of an IL-6 inhibitor, a TNF inhibitor, and an inhibitor of natural killer cells; a combination of an IL-6 inhibitor, an IL-8 inhibitor, and an IL18 inhibitor; a combination of an IL-6 inhibitor, an IL-8 inhibitor, and an inhibitor of natural killer cells; a combination of an IL-6 inhibitor, an IL-18 inhibitor, and an inhibitor of natural killer cells; a combination of an IL-6 inhibitor, a NLRP3 inhibitor, and a TNF inhibitor; a combination of an IL-6R inhibitor, a NLRP3 inhibitor, and a TNF inhibitor; a combination of an IL-6R inhibitor, a NLRP3 inhibitor, and an IL-8 inhibitor; a combination of an IL-6R inhibitor, a NLRP3 inhibitor, and an IL-18 inhibitor; a combination of an IL-6R inhibitor, a NLRP3 inhibitor, and an inhibitor of natural killer cells; a combination of an IL-6R inhibitor, a TNF inhibitor, and an IL-8 inhibitor; a combination of an IL-6R inhibitor, a TNF inhibitor, and an IL-18 inhibitor; a combination of an IL-6R inhibitor, an IL-8 inhibitor, and an IL18 inhibitor; a combination of an IL-6R inhibitor, an IL-8 inhibitor, and an inhibitor of natural killer cells; a combination of an NLRP3 inhibitor, a TNF inhibitor, and an IL-18 inhibitor; a combination of an NLRP3 inhibitor, a TNF inhibitor, and an inhibitor of natural killer cells; a combination of an NLRP3 inhibitor, an IL-8 inhibitor, and an IL18 inhibitor; a combination of an NLRP3 inhibitor, an IL-8 inhibitor, and an inhibitor of natural killer cells; a combination of an TNF inhibitor, an IL-8 inhibitor, and an IL18 inhibitor; a combination of an TNF inhibitor, an IL-8 inhibitor, and an inhibitor of natural killer cells; a combination of an IL-8 inhibitor, and an IL18 inhibitor, and an inhibitor of natural killer cells; or any suitable combination thereof of the earlier combinations. Any combination may be used. One skilled in the art can identify suitable combinations using routine methods.

In some embodiments, the anti-inflammatory agent comprises an IL-1 inhibitor. In some embodiments, an IL-1 inhibitor may be any protein or molecule capable of specifically preventing activation of cellular receptors to IL-1, which may result from any number of mechanisms. Exemplary mechanisms include, but are not limited to, downregulating IL-1 production, binding free IL-1, interfering with IL-1 binding to its receptor, interfering with formation of the IL-1 receptor complex (i.e., association of IL-1 receptor with IL-1 receptor accessory protein), and interfering with modulation of IL-1 signaling after binding to its receptor.

Certain interleukin-1 inhibitors include, but are not limited to, IL-1 binding proteins, including, but not limited to, soluble IL-1 receptors (see, e.g., U.S. Pat. Nos. 5,492,888, 5,488,032, and 5,464,937, 5,319,071, and 5,180,812, incorporated herein by reference); anti-IL-1 monoclonal antibodies (see, e.g., WO 9501997, WO 9402627, WO 9006371, U.S. Pat. No. 4,935,343, EP 364778, EP 267611 and EP 220063, incorporated herein by reference); IL-1 receptor accessory proteins and antibodies thereto (see, e.g., WO 96/23067 and WO 99/37773, incorporated herein by reference); inhibitors of interleukin-1 beta converting enzyme (ICE) or caspase 1 (see, e.g., WO 99/46248, WO 99/47545, and WO 99/47154, incorporated herein by reference), which may be used to inhibit IL-1 beta production and secretion; interleukin-1 beta protease inhibitors; and other compounds and proteins that block in vivo synthesis or extracellular release of IL-1.

Exemplary IL-1 inhibitors are disclosed, e.g., in U.S. Pat. Nos. 5,747,444; 5,359,032; 5,608,035; 5,843,905; 5,359,032; 5,866,576; 5,869,660; 5,869,315; 5,872,095; 5,955,480; 5,965,564; International (WO) patent applications 98/21957, 96/09323, 91/17184, 96/40907, 98/32733, 98/42325, 98/44940, 98/47892, 98/56377, 99/03837, 99/06426, 99/06042, 91/17249, 98/32733, 98/17661, 97/08174, 95/34326, 99/36426, 99/36415; European (EP) patent applications 534978 and 894795; and French patent application FR 2762514. The disclosures of all of the aforementioned references are hereby incorporated by reference for any purpose.

In some embodiments, the IL-1 inhibitor is an IL-1α inhibitor. In some embodiments, the IL-1α inhibitor is an anti-sense oligonucleotide against IL-1α, e.g., a RNAi molecules such as miRNA, siRNA, or shRNA. In some embodiments, the IL-1 inhibitor is an IL-1β inhibitor. In some embodiments, the IL-1β inhibitor is an anti-sense oligonucleotide against IL-1α, e.g., a RNAi molecules such as miRNA, siRNA, or shRNA. The nucleic acid sequences of IL-1A and IL-1B gene are known. One skilled in the art is able to design such anti-sense oligonucleotides using routine methods.

In some embodiments, the IL-1α inhibitor is an antibody against IL-1α, such as MABp1 (e.g., as described in Hong et al., Lancet Oncol. 2014 May; 15(6):656-66, incorporated herein by reference). In some embodiments, the IL-1α inhibitor is a protein that binds to IL-1α. In some embodiments, the protein that binds to IL-1α is a serum soluble interleukin-1 receptor type I (sIL-1RI, as described in Okamoto et al., J Clin Lab Anal. 2009; 23(3):175-8, incorporated herein by reference).

In some embodiments, the IL-1β inhibitor is an antibody against IL-1β, e.g., canakinumab, (e.g., as described in Ridker et al., N Engl J Med 2017; 377:1119-1131, incorporated herein by reference), gevokizumab (e.g., as described in Knickelbein et al., Am J Ophthalmol. 2016 December; 172:104-110, incorporated herein by reference), LY2189102 (e.g., as described in Sloan-Lancaster et al., Diabetes Care 2013 March; DC_121835, incorporated herein by reference), CYTO13 (e.g., as described in Dinarello et al., Nature Reviews Drug Discovery 11, 633-652, 2012, incorporated herein by reference). In some embodiments, the IL-1β inhibitor is a protein that binds to IL-1β. In some embodiments, the protein that binds to IL-1β is a serum soluble interleukin-1 receptor type II (sIL-1RII, e.g., as described in Jouvenne et al., Arthritis Rheum. 1998 June; 41(6):1083-9, incorporated herein by reference). In some embodiments, the IL-1β inhibitor inhibits caspase I, which is required in the production of IL-1β. In some embodiments, the caspase I inhibitor is VX-70 or VX-765 or belnacasan (e.g., as described in Boxer et al., ChemMedChem. 2010 May 3; 5(5): 730-738., incorporated herein by reference).

In some embodiments, the IL-1 inhibitor is a small molecule inhibitor selected from the group consisting of: suramin sodium, methotrexate-methyl-d3, methotrexate-methyl-d3 dimethyl ester, and diacerein. all of which are commercially available, e.g., from Santa Cruz Biotechnology, Inc., Texas, USA.

In some embodiments, the anti-inflammatory agent comprises an IL-1R inhibitor, e.g., an IL-1R antagonist. An “antagonist” is a type of receptor ligand or drug that blocks or dampens a biological response by binding to a receptor rather than provoking the response like an agonist. They are sometimes called blockers; examples include alpha blockers, beta blockers, and calcium channel blockers. In pharmacology, antagonists have affinity but no efficacy for their cognate receptors, and binding will disrupt the interaction and inhibit the function of an agonist or inverse agonist at receptors. Antagonists mediate their effects by binding to the active orthosteric (=right place) site or to allosteric (=other place) sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists achieve their potency by competing with endogenous ligands or substrates at structurally defined binding sites on receptors.

Naturally IL-1R antagonists include IL-1RA, IL-1RA variants, and IL-1RA derivatives, which are collectively termed “IL-1ra proteins.” Interleukin-1 receptor antagonist (IL-1ra) is a human protein that acts as a natural inhibitor of interleukin-1 and is a member of the IL-1 family, which includes IL-1α and IL-1. Certain receptor antagonists, including IL-1ra and variants and derivatives thereof, as well as methods of making and using them, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626, WO 94/20517; WO 96/22793; WO 97/28828; and WO 99/36541, which are incorporated herein by reference. In certain embodiments, an IL-1 receptor antagonist may be glycosylated. In certain embodiments, an IL-1 receptor antagonist may be non-glycosylated.

Three forms of IL-1ra and variants thereof are described in U.S. Pat. No. 5,075,222, incorporated herein by reference. Methods for isolating genes that code for the inhibitors, cloning those genes in suitable vectors, transforming and transfecting those genes into certain cell types, and expressing those genes to produce the inhibitors and known to those skilled in the art.

In some embodiments, the IL-1R inhibitor is an anti-sense oligonucleotide against IL-1R, e.g., a RNAi molecules such as miRNA, siRNA, or shRNA. The nucleic acid sequences of IL-1R gene is known. One skilled in the art is able to design such anti-sense oligonucleotides using routine methods.

In some embodiments, the IL-1R inhibitor is an antibody (e.g., a monoclonal antibody) against IL-1R. Exemplary IL-1 antibodies that may be used in accordance with the present disclosure include, without limitation: anakinra (e.g., as described in Mertens et al., Cochrane Database Syst Rev. 2009 Jan. 21; (1):CD005121, incorporated herein by reference), MEDI-8968 (e.g., as described in Dinarello et al, Nature Reviews Drug Discovery 11, 633-652, 2012, incorporated herein by reference), and AMG108 (e.g., as described in Cohen et al., Arthritis Res Ther. 2011 Jul. 29; 13(4):R125, incorporated herein by reference).

In some embodiments, the IL-1R inhibitor is an inhibitory protein or peptide. Such inhibitory protein or peptide include, without limitation: rilonacept, sIL-1RI (e.g., as described in Okamoto et al., J Clin Lab Anal. 2009; 23(3):175-8; and European patent EP 623674, incorporated herein by reference), and EBI-005 (e.g., as described in Kovalchin et al., Eye Contact Lens. 2017 Jul. 18. doi: 10.1097/ICL.0000000000000414, incorporated herein by reference).

In some embodiments, the anti-inflammatory agent comprises an IL-6 inhibitor. In some embodiments, the IL-6 inhibitor is an anti-sense oligonucleotide against IL-6, e.g., a RNAi molecules such as miRNA, siRNA, or shRNA. The nucleic acid sequences of IL-6 gene is known. One skilled in the art is able to design such anti-sense oligonucleotides using routine methods.

In some embodiments, the IL-6 inhibitor is an antibody against IL-6. Antibodies against IL-6 are known in the art and include BE-8 and CNT0328 (See e.g., Trikha et al., Clin Cancer Res 2003, 9: 4653 or US20090022726). As the IL-6-neutralizing antibodies, both polyclonal antibodies and monoclonal antibodies may be employed, and monoclonal antibodies are preferred. An example of the anti-IL-6 antibodies which have abilities to neutralize IL-6 is IG61 described in Japanese Laid-open Patent Application (Kokai) No. 3-139292 and in European Patent Publication 0 399 429 A1, although the IL-6-neutralizing antibody is not restricted to this antibody. IG61 was deposited in National Institute of Bioscience and Human-Technology, Agency of Industrial Science and Technology at 1-3, Higashi 1-chome, Tsukuba-shi, Ibaraki-ken, Japan, under accession number FERM BP-2878 on Apr. 27, 1990. Other non-limiting examples of IL-6 antibodies include siltuximab, sirukumab, clazakizumab, olokizumab, and elsilimomab. One skilled in the art is familiar with these IL-6 antibodies.

In some embodiments, the IL-6 inhibitor is a small molecule. Non-limiting, exemplary small molecule IL-6 inhibitors include: PGE1 and its derivatives, PGI2 and its derivatives, and cyclophosphamide.

In some embodiments, the anti-inflammatory agent comprises an IL-6R inhibitor (e.g., an IL-6R antagonist). In some embodiments, the IL-6R inhibitor is an anti-sense oligonucleotide against IL-6R, e.g., a RNAi molecules such as miRNA, siRNA, or shRNA. The nucleic acid sequences of IL-6R gene is known. One skilled in the art is able to design such anti-sense oligonucleotides using routine methods.

In some embodiments, the IL-6R inhibitor is an IL-6R antibody. Antibodies against IL-6R are known in the art and include PM1 (Hirata et al., J Immunol 143, 2900, 1986, incorporated herein by reference), AUK12-20, AUK64-7, AUK146-15 (WO92/19759), MRA (U.S. Pat. No. 5,888,510), AB-227-NA and Tocilizumab (See e.g., Hashizume, Rheumat Int 2009 Jul. 29, epub, incorporated herein by reference). These antibodies are capable of neutralizing IL-6 signaling via binding to either IL-6 or its receptor. Such antibodies can also be prepared via routine technologies. In some embodiments, the IL-6R antibody is sarilumab (e.g., as described in Raimondo et al., Drug Des Devel Ther. 2017 May 24; 11:1593-1603, incorporated herein by reference).

In some embodiments, the anti-inflammatory agent comprises a NLRP3 inhibitor. In some embodiments, the NLRP3 inhibitor is an anti-sense oligonucleotide against NLRP3, e.g., a RNAi molecules such as miRNA, siRNA, or shRNA. The nucleic acid sequences of NLRP3 gene is known. One skilled in the art is able to design such anti-sense oligonucleotides using routine methods.

Other NLRP3 inhibitors are described in the art, e.g., in Shao et al., Front Pharmacol. 2015; 6: 262, incorporated herein by reference. Non-limiting examples of NLRP3 inhibitors include: colchicine, MCC950, CY-09, ketone metabolite beta-hydroxubutyrate (BHB), a type I interferon, resveratrol, arglabin, CB2R, glybenclamide, isoliquiritigenin, Z-VAD-FMK, and microRNA-223. Several of the NLRP3 inhibitors described herein, e.g., glybenclamide, isoliquiritigenin, and Z-VAD-FMK are commercially available, e.g., from Invivogen Inc. (California, USA).

In some embodiments, the anti-inflammatory agent comprises a TNF inhibitor, e.g., TNFα. In some embodiments, the TNFα inhibitor is an anti-sense oligonucleotide against TNFα, e.g., a RNAi molecules such as miRNA, siRNA, or shRNA. The nucleic acid sequences of TNFα gene is known. One skilled in the art is able to design such anti-sense oligonucleotides using routine methods.

In some embodiments, TNF inhibitors may act by at least one of downregulating or inhibiting TNF production, binding free TNF, interfering with TNF binding to its receptor, and interfering with modulation of TNF signaling after binding to its receptor. Examples of TNF inhibitors include, without limitation, solubilized TNF receptors, including, but not limited to, soluble tumor necrosis factor receptor type I (sTNF-RI; also called the p55 receptor), soluble tumor necrosis factor receptor type II (also called the p75 receptor), and Enbrel™; antibodies to TNF, including, but not limited to, Remicade™ and D2E7 (see, e.g., U.S. Pat. Nos. 6,090,382 and 6,258,562); antibodies to TNF receptor; sTNF-RI (see, e.g., WO 98/24463), etanercept (Enbrel™), Avakine™; inhibitors of TNF-α converting enzyme (TACE); and other molecules that affect TNF activity.

Exemplary TNF-α inhibitors are described in the art, e.g., in European patent applications EP 308 378; EP 422 339; EP 393 438; EP 398 327; EP 412 486; EP 418 014, EP 417 563, EP 433 900; EP 464 533; EP 512 528; EP 526 905; EP 568 928; EP 607 776, which describes the use of leflunomide for inhibition of TNF-α; EP 663 210; EP 542 795; EP 818 439; EP 664 128; EP 542 795; EP 741707; EP 874 819; EP 882 714; EP 880 970; EP 648 783; EP731791; EP895988; EP550376; EP882714; EP853083; EP550376; EP943 616; EP 939 121; EP 614 984; EP 853 083; U.S. Pat. Nos. 5,136,021; 5,929,117; 5,948,638; 5,807,862; 5,695,953; 5,834,435; 5,817,822; 5,830,742; 5,834,435; 5,851,556; 5,853,977; 5,359,037, 5,512,544; 5,695,953; 5,811,261; 5,633,145; 5,863,926; 5,866,616; 5,641,673; 5,869,677; 5,869,511; 5,872,146; 5,854,003; 5,856,161; 5,877,222; 5,877,200; 5,877,151; 5,886,010; 5,869,660; 5,859,207; 5,891,883; 5,877,180; 5,955,480; 5,955,476; 5,955,435; 5,994,351; 5,990,119; 5,952,320; 5,962,481; International patent applications WO 90/13575, WO 91/03553, WO 92/01002, WO 92/13095, WO 92/16221, WO 93/07863, WO 93/21946, WO 93M19777, WO 95/34326, WO 96/28546, WO 98/27298, WO 98/30541, WO 96/38150, WO 96/38150, WO 97/18207, WO 97/15561, WO 97/12902, WO 96/25861, WO 96/12735, WO 96/11209, WO 98/39326, WO 98/39316, WO 98/38859, WO 98/39315, WO 98/42659, WO 98/39329, WO 98/43959, WO 98/45268, WO 98/47863, WO 96/33172, WO 96/20926, WO 97/37974, WO 97/37973, WO 97/47599, WO 96/35711, WO 98/51665, WO 98/43946, WO 95/04045, WO 98/56377, WO 97/12244, WO 99/00364, WO 99/00363, WO 98/57936, WO 99/01449, WO 99/01139, WO 98/56788, WO 98/56756, WO 98/53842, WO 98/52948, WO 98/52937, WO 99/02510, WO 97/43250, WO 99/06410, WO 99/06042, WO 99/09022, WO 99/08688, WO 99/07679, WO 99/09965, WO 99/07704, WO 99/06041, WO 99/37818, WO 99/37625, WO 97/11668, WO 99/50238, WO 99/47672, WO 99/48491; Japanese patent applications 10147531, 10231285, 10259140, and 10130149, 10316570, 11001481, and 127,800/1991; German application no. 19731521; and British application Nos. 2 218 101, 2 326 881, 2 246 569. The disclosures of all of the aforementioned references are hereby incorporated by reference for any purpose.

In some embodiments, the TNF inhibitor is a TNF antibody, e.g., without limitation, infliximab, adalimumab, certolizumab pegol, and golimumab. In some embodiments, the TNF inhibitor is etanercept (Enbrel). Non-limiting examples of small molecule TNF inhibitors include: thalidomide, lenalidomide, pomalidomide, a xanthine derivative, bupropion, 5-HT2A agonist hallucinogens (e.g., (R)-DOI, TCB-2, LSD and LA-SS-Az).

In some embodiments, the anti-inflammatory agent comprises an IL-8 inhibitor. In some embodiments, the IL-8 inhibitor is an anti-sense oligonucleotide against IL-8, e.g., a RNAi molecules such as miRNA, siRNA, or shRNA. The nucleic acid sequences of IL-8 gene is known. One skilled in the art is able to design such anti-sense oligonucleotides using routine methods.

In some embodiments, the IL-8 inhibitor is an antibody against IL-8, e.g., without limitation, HuMab-10F8 as described in Skov et al., J Immunol. 2008 Jul. 1; 181(1):669-79, incorporated herein by reference. In some embodiments, the IL-8 inhibitor is Reparixin, e.g., as described in Leitner et al., Int J Immunopathol Pharmacol. 2007 January-March; 20(1):25-36, incorporated herein by reference. Non-limiting examples of small molecule IL-8 inhibitors include: curcumin, antileukinate, macrolide (e.g., as described in Kohyama et al., Antimicrob. Agents Chemother. April 1999 vol. 43 no. 4 907-911, incorporated herein by reference), and a trifluoroacetate salt.

In some embodiments, the anti-inflammatory agent comprises an IL-18 inhibitor. In some embodiments, the IL-18 inhibitor is an anti-sense oligonucleotide against IL-18, e.g., a RNAi molecules such as miRNA, siRNA, or shRNA. The nucleic acid sequences of IL-18 gene is known. One skilled in the art is able to design such anti-sense oligonucleotides using routine methods.

Exemplary IL-18 inhibitors include, but are not limited to, antibodies that bind to IL-18; antibodies that bind to IL-18R; antibodies that bind to IL-18RAcP; IL-18 bp; IL-18R fragments (e.g., a solubilized extracellular domain of the IL-18 receptor); peptides that bind to IL-18 and reduce or prevent its interaction with IL-18R; peptides that bind to IL-18R and reduce or prevent its interaction with IL-18 or with IL-18RAcP, peptides that bind to IL-18RAcP and reduce or prevent its interaction with IL-18R; and small molecules that reduce or prevent IL-18 production or the interaction between any of IL-18, IL-18R, and IL-18RAcP.

Certain IL-18 inhibitors are described, e.g., in U.S. Pat. No. 5,912,324, issued Jul. 14, 1994; EP 0 962 531, published Dec. 8, 1999; EP 712 931, published Nov. 15, 1994; U.S. Pat. No. 5,914,253, issued Jul. 14, 1994; WO 97/24441, published Jul. 10, 1997; U.S. Pat. No. 6,060,283, issued May 9, 2000; EP 850 952, published Dec. 26, 1996; EP 864 585, published Sep. 16, 1998; WO 98/41232, published Sep. 24, 1998; U.S. Pat. No. 6,054,487, issued Apr. 25, 2000; WO 99/09063, published Aug. 14, 1997; WO 99/22760, published Nov. 3, 1997; WO 99/37772, published Jan. 23, 1998; WO 99/37773, published Mar. 20, 1998; EP 0 974 600, published Jan. 26, 2000; WO 00/12555, published Mar. 9, 2000; Japanese patent application JP 111,399/94, published Oct. 31, 1997; Israel patent application IL 121554 A0, published Feb. 8, 1998; which are incorporated herein by reference.

In some embodiments, the IL-18 inhibitor is an IL-18 binding protein, e.g., as described in Dinarello et al., Front Immunol. 2013; 4: 289, incorporated herein by reference. In some embodiments, the IL-18 inhibitor is a small molecule, such as the NSC201631, NSC61610, and NSC80734 described in Krumm et al., Scientific Reports 7, Article number: 483, 2017, incorporated herein by reference.

In some embodiments, the anti-inflammatory agent comprises an inhibitor of natural killer cells. In some embodiments, the inhibitor of natural killer cells is an antibody (e.g., the MKp46 antibody described in Yossef et al., The Journal of Immunology, Vol. 192, Issue 1 Supplement 1 May 2014, incorporated herein by reference). In some embodiments, the inhibitor of natural killer cells is a viral major histocompatibility complex (MHC) class I homologue (e.g., as described in Farrell et al., Nature volume 386, pages 510-514, 1997, incorporated herein by reference). In some embodiments, the inhibitor of natural killer cells is a dietary lipid (e.g., as described in Yaqoob et al., Immunology Letters, Volume 41, Issues 2-3, July 1994, Pages 241-247, incorporated herein by reference). One skilled in the art is able to choose appropriate inhibitors of natural killer cells.

In some embodiments, the anti-inflammatory agent comprises any other cytokine inhibitors described in the art, e.g., in PCT Application Publications WO2007075896, WO2008021388, WO2007056016, and WO2007056016, and in US Patent Application Publication US20040033535, incorporated herein by reference. In some embodiments, the anti-inflammatory agent comprises methotrexate. In some embodiments, the anti-inflammatory agent comprises arhalofenate, e.g., as described in Poiley et al., Arthritis & Rheumatology, Vol. 68, No. 8, August 2016, pp 2027-2034, incorporated herein by reference.

The methods described herein are combination therapy methods. The subject is administered an anti-inflammatory agent and a lipid lowering agent. A “lipid lowering agent” refers to an agent that reduces the level of one or more lipids (e.g., by at least 20%, at least 30%, at least 40%, at least 50%, at least 60%, at least 70%, at least 80%, or more) in a subject (e.g., a subject who has or is at risk of developing a cardiovascular disease). Examples of lipids whose level may be reduced by the lipid lowering agent described herein include, without limitation: cholesterol (e.g., total cholesterol), LDL-C, very low density lipoprotein cholesterol (VLDL-C), non-high density lipoprotein cholesterol (non-HDL-C), and triglycerides. In important embodiments, the lipid is LDL-C. In some embodiments, the lipid lowering agent increases the level of high density lipoprotein cholesterol (HDL-C) in a subject (e.g., by e.g., by at least 20%, at least 30%, at least 40%, at least 50%, at least 60%, at least 70%, at least 80%, at least 90%, at least 100%, 2-fold, 5-fold, 10-fold, or more).

Non-limiting examples of lipid lowering agents include, without limitation: HMG-CoA reductase inhibitors (e.g., statins), a proprotein convertase subtilisin/kexin type 9 (PCSK9) inhibitors, other lipid lowering agents, and/or combinations thereof. In some embodiments, the lipid lowering agent of the present disclosure comprises a HMG-CoA reductase inhibitor. By reducing the amount of cholesterol synthesized by the cell, through inhibition of the HMG-CoA reductase gene, a cycle of events is initiated that culminates in the increase of LDL-C uptake by liver cells. As LDL-C uptake is increased, total cholesterol and LDL-C levels in the blood decrease.

In some embodiments, the HMG-CoA reductase inhibitor is a statin. Non-limiting examples of statins include: simvastatin (Zocor), lovastatin (Mevacor), pravastatin (Pravachol), fluvastatin (Lescol), atorvastatin (Lipitor), cerivastatin. (Baycol), rosuvastatin (Crestor), pitivastatin and numerous others described in U.S. Pat. Nos. 4,444,784, 4,231,938, 4,346,227, 4,739,073, 5,273,995, 5,622,985, 5,135,935, 5,356,896, 4,920,109, 5,286,895, 5,262,435, 5,260,332, 5,317,031, 5,283,256, 5,256,689, 5,182,298, 5,369,125, 5,302,604, 5,166,171, 5,202,327, 5,276,021, 5,196,440, 5,091,386, 5,091,378, 4,904,646, 5,385,932, 5,250,435, 5,132,312, 5,130,306, 5,116,870, 5,112,857, 5,102,911, 5,098,931, 5,081,136, 5,025,000, 5,021,453, 5,017,716, 5,001,144, 5,001,128, 4,997,837, 4,996,234, 4,994,494, 4,992,429, 4,970,231, 4,968,693, 4,963,538, 4,957,940, 4,950,675, 4,946,864, 4,946,860, 4,940,800, 4,940,727, 4,939,143, 4,929,620, 4,923,861, 4,906,657, 4,906,624 and 4,897,402.

Non-limiting examples of statins already approved for use in humans include atorvastatin, cerivastatin, fluvastatin, pravastatin, simvastatin and rosuvastatin. HMG-CoA reductase inhibitors are also described in Drugs and Therapy Perspectives (May 12, 1997), 9: 1-6; Chong (1997) Pharmacotherapy 17:1 157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368, each of which is incorporated herein by reference. Another statin drug of note is compound 3a (S-4522) described in in Watanabe (1997) Bioorganic and Medicinal Chemistry 5: 437-444, incorporated herein by reference.

In some embodiments, the lipid lowering agent comprises a proprotein convertase subtilisin/kexin type 9 (PCSK9) inhibitor. “proprotein convertase subtilisin/kexin type 9 (PCSK9)” is an enzyme encoded by the PCSK9 gene in humans. PCSK9 binds to the receptor for low-density lipoprotein (LDL) particles. In the liver, the LDL receptor removes LDL particles from the blood through the endocytosis pathway. When PCSK9 binds to the LDL receptor, the receptor is channeled towards the lysosomal pathway and broken down by proteolytic enzymes, limiting the number of times that a given LDL receptor is able to uptake LDL particles from the blood. Inhibiting PCSK9 level or activity may lead to more LDL receptors being recycled and present on the surface of the liver cells, and will remove more LDL cholesterol from the blood, in turn lowering blood cholesterol levels.

Various therapeutic approaches to the inhibition of PSCK9 have been proposed, including: inhibition of PSCK9 synthesis by gene silencing agents, e.g., RNAi; inhibition of PCSK9 binding to LDL-R by monoclonal antibodies, small peptides or adnectins; and inhibition of PCSK9 autocatalytic processing by small molecule inhibitors. These strategies have been described in Hedrick et al., Curr Opin Investig Drugs 2009; 10:938-46; Hooper et al., Expert Opin Biol Ther, 2013; 13:429-35; Rhainds et al., Clin Lipid, 2012; 7:621-40; Seidah et al; Expert Opin Ther Targets 2009; 13:19-28; and Seidah et al., Nat Rev Drug Discov 2012; 11:367-83, each of which are incorporated herein by reference.

A “PCSK9 inhibitor” refers to an agent that reduces the level or activity of PCSK9 (e.g., in a subject). In some embodiments, the PCSK9 inhibitor reduces the expression of PCSK9 (e.g., by at least 20%, at least 30%, at least 40%, at least 50%, at least 60%, at least 70%, at least 80%, at least 90%, or 100%). In some embodiments, the PCSK9 inhibitor reduces the activity of PSCK9 (e.g., by at least 20%, at least 30%, at least 40%, at least 50%, at least 60%, at least 70%, at least 80%, at least 90%, or 100%). In some embodiments, the PCSK9 inhibitor is selected from the group consisting of: natural PCSK9 inhibitors, PCSK9 antibodies, antisense nucleic acids, peptide inhibitors, PCSK9 vaccines, and small molecule inhibitors.

In some embodiments, the PCSK9 inhibitor is a natural PCSK9 inhibitor. A “natural PCSK9 inhibitor” refers to a naturally occurring molecule (e.g., in plants or in a mammal) that has inhibitory activity against PCSK9. For example, plant alkaloid berberine inhibits the transcription of the PCSK9 gene in immortalized human hepatocytes in vitro (e.g., as described in Li et al., The Journal of Biological Chemistry. 284 (42): 28885-95, 2009, incorporated herein by reference) and lowers serum PCSK9 in mice and hamsters in vivo (e.g., as described in Dong et al., The Journal of Biological Chemistry. 290 (7): 4047-58, 2015, incorporated herein by reference). In another example, Annexin A2, which is an endogenous protein, inhibits PCSK9 activity (e.g., as described in Seidah et al., PLoS ONE. 7 (7): e41865, 2012, incorporated herein by reference). In some embodiments, the PCSK9 inhibitor is adnectin (BMS-962476, as described in Mitchell et al., J Pharmacol Exp Ther. 2014 August; 350(2):412-24, incorporated herein by reference).

In some embodiments, the PCSK9 inhibitor is a PCSK9 antibody. Non-limiting examples of PCSK9 antibodies include: alirocumab (Praluent®, as described in Robinson et al., N Engl J Med 2015; 372:1489-1499, 2015, incorporated herein by reference), evolocumab (Repatha, e.g., as described in Sabatine et al., N Engl J Med 2017; 376:1713-1722, 2017, incorporated herein by reference), 1D05-IgG2 (e.g., as described in Ni et al., J Lipid Res. 2011 January; 52(1):78-86m incorporated herein by reference), RG-7652 (e.g., as described in Baruch et al., Am J Cardiol. 2017 May 15; 119(10):1576-1583, incorporated herein by reference), LY3015014 (e.g., as described in Eur Heart J. 2016 May 1; 37(17):1360-9, incorporated herein by reference), and bococizumab (e.g., as described in Ridker et al., N Engl J Med 2017; 376:1527-1539, incorporated herein by reference). The examples illustrated herein are not intended to be limiting. Any PCSK9 antibodies that inhibit its activity may be used in accordance with the present disclosure.

In some embodiments, the PCSK9 inhibitor is an antisense nucleic acid. In some embodiments, the anti-sense nucleic acid is an RNAi molecule (microRNA, siRNA, shRNA, dsRNA and other small interfering nucleic acid-based molecules known in the art. The nucleic acid sequence of PCSK9 is known in the art (e.g., human PCSK9, NCBI gene ID: 255738). One skilled in the art is familiar with how to make and use antisense nucleic acids targeting the PCSK9 gene. In some embodiments, the RNAi molecule that inhibits PCSK9 expression is inclisiran (e.g., as described in Ray et al., N Engl J Med 2017; 376:1430-1440, incorporated herein by reference) or ALN-PCS (e.g., as described in Fitzgerald et al., N Engl J Med. 2017 Jan. 5; 376(1):41-51, incorporated herein by reference).

In some embodiments, the PCSK9 inhibitor is a peptide inhibitor. In some embodiments, the peptide inhibitor is a peptide that mimics an EGFa domain of low-density lipoprotein receptor (LDL-R) (e.g., as described in Kwon et al., PNAS 2008 February, 105 (6) 1820-1825; and Schroeder et al., Chemistry & Biology, Volume 21, Issue 2, 20 Feb. 2014, Pages 284-294, incorporated herein by reference). In some embodiments, the peptide inhibitor is the Pep2-8 as described in Zhang et al., The Journal of Biological Chemistry 289, 942-955, incorporated herein by reference).

In some embodiments, the PCSK9 inhibitor is a small molecule. In some embodiments, the small molecule PCSK9 inhibitor is PF-06446846 (e.g., as described in Lintner et al., PLoS Biol 15(3): e2001882, incorporated herein by reference). In some embodiments, the small molecule PCSK9 inhibitor is an inhibitor of cholesteryl ester transfer protein (CETP), such as anacetrapib (e.g., as described in Barter et al., J Lipid Res. 2015 November; 56(11): 2045-2047, incorporated herein by reference) or K-312 (e.g., as described in Miyosawa et al., Am J Physiol Endocrinol Metab. 2015 Jul. 15; 309(2):E177-90, incorporated herein by reference). Other examples of small molecule PCSK9 inhibitors are described in Petersen et al., Cell Chemical Biology, Volume 23, Issue 11, p1362-1371, 2016 and Halford et al., Chemical & Engineering News, Volume 94 Issue 44 1 p. 12, 2016, incorporated herein by reference.

In some embodiments, the PCSK9 inhibitor is a PCSK9 vaccine. In some embodiments, the PCSK9 vaccine comprises an antigenic peptide from PCSK9. For example, the PCSK9 vaccine may be the AT04A vaccine described in Landlinger et al. (European Heart Journal, Volume 38, Issue 32, 21 Aug. 2017, Pages 2499-2507, incorporated herein by reference). In some embodiments, the PSCK9 vaccine may be a virus-like particle-peptide vaccine (e.g., the PCSK9Qβ-003 vaccine described in Pan et al., Scientific Reports volume 7, Article number: 12534 (2017), incorporated herein by reference).

Any other known PCSK9 inhibitory strategies may be used in accordance with the present disclosure. For example, PCSK9 genes may be modified to result in a non-functional PCSK9 variant in the subject, thus inhibit its activity. Numerous PCSK9 variants are described, e.g., in PCT Publication Nos. WO2001031007, WO2001057081, WO2002014358, WO2001098468, WO2002102993, WO2002102994, WO2002046383, WO2002090526, WO2001077137, and WO2001034768; US Publication Nos. US 2004/0009553 and US 2003/0119038, and European Publication Nos. EP 1 440 981, EP 1 067 182, and EP 1 471 152, each of which are incorporated herein by reference.

Several mutant forms of PCSK9 are well characterized, including S127R, N157K, F216L, R218S, and D374Y, with S127R, F216L, and D374Y being linked to autosomal dominant hypercholesterolemia (ADH). See Benjannet et al. (J. Biol. Chem., 279(47):48865-48875 (2004)); Rashid et al., PNAS, 102(15):5374-5379 (2005); Abifadel et al., 2003 Nature Genetics 34:154-156; Timms et al., 2004 Hum. Genet. 114:349-353; Leren, 2004 Clin. Genet. 65:419-422; Cohen et al., 2006 N. Engl. J. Med. 354:1264-1272; Lalanne et al. (J. Lipid Research, 46:1312-1319 (2005); each of which are incorporated herein by reference.

In some embodiments, the lipid lowering agent comprises one or more (e.g., 1, 2, 3, or more) HMG-CoA reductase inhibitors (e.g., statins) and one or more (e.g., 1, 2, 3, or more) PSCK9 inhibitors known in the art or described herein. For example, the lipid lowering agent may comprise one or more (e.g., 1, 2, 3, or more) of simvastatin, lovastatin, pravastatin, fluvastatin, atorvastatin, cerivastatin, rosuvastatin, and pitivastatin, and one or more (e.g., 1, 2, 3, or more) of berberine, annexin A2, adnectin, PF-06446846, anacetrapib, K-312, alirocumab, evolocumab, 1D05-IgG2, RG-7652, LY3015014, bococizumab, inclisiran, ALN-PCS, and PCSK9 vaccines. All possible combinations are contemplated herein.

In some embodiments, the lipid lowering agent described herein further comprises one or more of other agents that has lipid-lowering effect, e.g., without limitation: fibric acid derivatives (fibrates), bile acid sequestrants or resins, nicotinic acid agents, cholesterol absorption inhibitors, acyl-coenzyme A: cholesterol acyl transferase (ACAT) inhibitors, cholesteryl ester transfer protein (CETP) inhibitors, LDL receptor antagonists, farnesoid X receptor (FXR) antagonists, sterol regulatory binding protein cleavage activating protein (SCAP) activators, microsomal triglyceride transfer protein (MTP) inhibitors, squalene synthase inhibitors, and peroxisome proliferation activated receptor (PPAR) agonists.

Non-limiting examples of fibric acid derivatives include: gemfibrozil (Lopid), fenofibrate (Tricor), clofibrate (Atromid) and bezafibrate. Non-limiting examples of bile acid sequestrants or resins include: colesevelam (WelChol), cholestyramine (Questran or Prevalite) and colestipol (Colestid), DMD-504, GT-102279, HBS-107 and S-8921. Non-limiting examples of nicotinic acid agents include: niacin and probucol. Examples of cholesterol absorption inhibitors include but are not limited to ezetimibe (Zetia). Non-limiting examples of ACAT inhibitors include: Avasimibe, CI-976 (Parke Davis), CP-113818 (Pfizer), PD-138142-15 (Parke Davis), F1394, and numerous others described in U.S. Pat. Nos. 6,204,278, 6,165,984, 6,127,403, 6,063,806, 6,040,339, 5,880,147, 5,621,010, 5,597,835, 5,576,335, 5,321,031, 5,238,935, 5,180,717, 5,149,709, and 5,124,337. Non-limiting examples of CETP inhibitors include: Torcetrapib, CP-529414, CETi-I, JTT-705, and numerous others described in U.S. Pat. Nos. 6,727,277, 6,723,753, 6,723,752, 6,710,089, 6,699,898, 6,696,472, 6,696,435, 6,683,099, 6,677,382, 6,677,380, 6,677,379, 6,677,375, 6,677,353, 6,677,341, 6,605,624, 6,586,448, 6,521,607, 6,482,862, 6,479,552, 6,476,075, 6,476,057, 6,462,092, 6,458,852, 6,458,851, 6,458,850, 6,458,849, 6,458,803, 6,455,519, 6,451,830, 6,451,823, 6,448,295, 5,512,548. One non-limiting example of an FXR antagonist is Guggulsterone. One non-limiting example of a SCAP activator is GW532 (GlaxoSmithKline). Non-limiting examples of MTP inhibitors include: Implitapide and R-103757. Non-limiting examples of squalene synthase inhibitors include: zaragozic acids. Non-limiting examples of PPAR agonists include: GW-409544, GW-501516, and LY-510929.

In some embodiments, the method of treating cardiovascular disease is further combined with other therapies for reducing the risk of a future cardiovascular event, e.g., without limitation: diet and/or exercise and/or therapies with: anti-lipemic agents, anti-inflammatory agents, anti-thrombotic agents, fibrinolytic agents, anti-platelet agents, direct thrombin inhibitors, glycoprotein Ib/Ia receptor inhibitors, agents that bind to cellular adhesion molecules and inhibit the ability of white blood cells to attach to such molecules (e.g., anti-cellular adhesion molecule antibodies), alpha-adrenergic blockers, beta-adrenergic blockers, cyclooxygenase-2 inhibitors, angiotensin system inhibitor, anti-arrhythmics, calcium channel blockers, diuretics, inotropic agents, vasodilators, vasopressors, thiazolidinediones, cannabinoid-1 receptor blockers and/or any combinations thereof.

In some aspects, the present disclosure provides strategies of treating a cardiovascular disease by reducing inflammation and reducing lipid level simultaneously using a bispecific antibody that targets both a proinflammatory cytokine and PCSK9. In some embodiments, the method comprises administering to a subject in need thereof a therapeutically effective amount of a bispecific antibody comprising a first antigen-binding domain that binds an proinflammatory cytokine and a second antigen-binding domain that binds PCSK9.

A “bispecific antibody” is an antibody with dual antigen binding specificities. Bispecific antibodies can be formed by joining two antigen binding domains that have different binding specificities. As such, a bispecific antibody comprises a first antigen binding domain that binds a first antigen and a second antigen binding domain that binds a second antigen that is different from the first antigen.

An “antigen binding domain” is also termed herein as an “antigen binding fragment” or “antigen binding portion” and refers to a polypeptide having specific binding affinity for an epitope of an antigen. In some embodiments, such polypeptide is encoded by immunoglobulin genes. Non-limiting examples of immunoglobulin genes include the kappa, lambda, alpha, gamma, delta, epsilon and mu constant region genes, as well as the myriad immunoglobulin variable region genes. The immunoglobulins may exist in a variety of forms besides antibodies; including, for example, Fv, Fab, and F(ab)2, and single chains (e.g., as described in Huston, et al., Proc. Nat. Acad. Sci. U.S.A., 85:5879-5883 (1988) and Bird, et al., Science, 242:423-426 (1988), which are incorporated herein by reference). Other examples of antigen-binding domains include T-cell antigen receptors and the CD4 protein, which binds to an epitope on MHC protein. In addition to the naturally-occurring forms of immunoglobulin chains, antigen-binding domains can be designed and manufactured using various recombinant DNA techniques well known to those skilled in the art.

Bispecific antibodies may be in various formats. In some embodiments, the bispecific antibody is an Ig-G like molecule. That is, the bispecific antibody comprises a first antigen-binding domain, a second antigen-binding domain and a common fragment crystallizable region (Fc region). In some embodiments, the bispecific antibody is a monoclonal bispecific antibody. Monoclonal bispecific antibodies retain the traditional monoclonal antibody (mAb) structure of two antigen binding domains and one Fc region, except the two antigen binding domains bind different antigens. The most common types of monoclonal bispecific antibodies are called trifunctional antibodies, as they have three unique binding sites on the antibody: the two Fab regions, and the Fc region. Each antigen binding domain (e.g., a heavy and light chain pair) of a monoclonal bispecific antibody is derived from a unique monoclonal antibody. The Fc region made from the two heavy chains forms the third binding site that binds to cell surface Fc receptors. These bispecific monoclonal antibodies are often manufactured with the quadroma, or the hybrid hybridoma method.

In some embodiments, the bispecific antibody is non-IgG-like. There are other bispecific antibodies that lack an Fc region entirely. Non-IgG-like bispecific antibodies include chemically linked Fabs, consisting of only the Fab regions, various types of bivalent and trivalent single-chain variable fragments (scFvs), and fusion proteins mimicking the variable domains of two antibodies. One example of a non-IgG like bispecific antibody is the bispecific T-cell engagers (BiTEs, e.g., as described in Yang et al, International Journal of Molecular Sciences. 18 (1): 48, 2016; Baeuerle et al., Cancer Res. 69 (12): 4941-4944, 2009; and Wozniak-Knopp et al., Protein Engineering Design and Selection. 23 (4): 289-297, 2010, incorporated herein by reference).

Bispecific antibodies may be produced by various methods known to those skilled in art. The two antigen-binding domains of the bispecific antibody may be derived from an antibody against a proinflammatory cytokine and against an antibody against PCSK9. “Derive from” means to use the antigen-binding domain of an antibody to a proinflammatory cytokine described herein as the first antigen binding domain of the bispecific antibody, and to use the antigen-binding domain of a PCSK9 antibody as the second antigen binding domain of the bispecific antibody. The two antigen-binding domains may be attached to each other by chemical cross-linking, by linking through a pair of epitopes that interact with each other (e.g., leucine zipper), by hybrid-hybridomas (Milstein and Cuello, (1984) Immunol. Today 5:299) or transfectomas, or by disulfide exchange at the hinge region. One skilled in the art is familiar with methods of producing the bispecific antibody.

In some embodiments, the proinflammatory cytokine targeted by the first antigen-binding domain may be any of the proinflammatory cytokines described herein, e.g., without limitation, IL-1, IL-1 receptor (IL-1R), IL-6, IL-6 receptor (IL-6R), NLRP3, TNF, IL-8, or IL-18.

In some embodiments, the bispecific antibody comprises a first antigen-binding domain that binds IL-1 (e.g., IL-1α or IL-1β) and a second antigen-binding domain that binds PCSK9. In some embodiments, the first antigen-binding domain binds to IL-1α. In some embodiments, the first antigen-binding domain is derived from an IL-1α antibody (e.g., without limitation, MABp1). In some embodiments, the first antigen-binding domain binds to IL-1β. In some embodiments, the first antigen-binding domain is derived from an IL-1β antibody (e.g., without limitation, canakinumab, gevokizumab, diacerein, or LY2189102).

In some embodiments, the first antigen-binding domain binds to IL-1R. In some embodiments, the first antigen-binding domain is derived from an IL-1R antibody (e.g., without limitation, MEDI-8968 or AMG108).

In some embodiments, the first antigen-binding domain binds to IL-6. In some embodiments, the first antigen-binding domain is derived from an IL-6 antibody (e.g., without limitation, siltuximab, sirukumab, clazakizumab, olokizumab, or elsilimomab).

In some embodiments, the first antigen-binding domain binds to IL-6R. In some embodiments, the first antigen-binding domain is derived from an IL-6R antibody (e.g., without limitation, tocilizumab, sarilumab, PM1, AUK12-20, AUK64-7, AUK146-15, or AB-227-NA).

In some embodiments, the first antigen-binding domain binds to NLRP3. In some embodiments, the first antigen-binding domain is derived from an NLRP3 antibody.

In some embodiments, the first antigen-binding domain binds to TNF. In some embodiments, the first antigen-binding domain is derived from a TNF antibody (e.g., without limitation, infliximab, adalimumab, certolizumab pegol, golimumab, or etanercept (Enbrel)).

In some embodiments, the first antigen-binding domain binds to IL-8. In some embodiments, the first antigen-binding domain is derived from an IL-8 antibody (e.g., without limitation, HuMab-10F8).

In some embodiments, the first antigen-binding domain binds to IL-18. In some embodiments, the first antigen-binding domain is derived from an IL-18 antibody.

In some embodiments, the second antigen-binding domain is derived from a PCSK9 antibody, (e.g., without limitation, alirocumab, evolocumab, 1D05-IgG2, RG-7652, LY3015014, or bococizumab).

In some embodiments, the subject may be further administered therapeutically effective amount of a HMG-CoA reductase inhibitor in addition to the bispecific antibody described herein. In some embodiments, the HMG-CoA reductase inhibitor is a statin (e.g., without limitation, simvastatin, lovastatin, pravastatin, fluvastatin, atorvastatin, cerivastatin, rosuvastatin, or pitivastatin). In some embodiments, the level or activity of a proinflammatory cytokine is reduced in the subject received treatment with the lipid lowering agent and the anti-inflammatory agent described herein, relative to before receiving the treatment. “Reduce the level or activity of a proinflammatory cytokine” means that the level or activity of the cytokine (e.g., IL-1, IL-6, TNF, IL-8, or IL-18) is reduced by at least 20% lower when the composition is administered to the subject, compared to without the composition. For example, the level or activity of the cytokine (e.g., IL-1, IL-6, TNF, IL-8, or IL-18) may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the level or activity of the cytokine (e.g., IL-1, IL-6, TNF, IL-8, or IL-18) is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment. The activity of a proinflammatory cytokine may be reflected in the magnitude of the signaling pathway. One skilled in the art can assess the activity of a proinflammatory cytokine using routine methods.

In some embodiments, the level or activity of C-reactive protein reduced in the subject received treatment with the lipid lowering agent and the anti-inflammatory agent described herein, relative to before receiving the treatment. “C-reactive protein (CRP)” is a substance produced by the liver that increases in the presence of inflammation in the body. An elevated C-reactive protein level is identified with blood tests and is considered a non-specific “marker” for disease.

In some embodiments, a subject having a cardiovascular disease or is at risk of developing a cardiovascular disease has a CRP level that is at least 20% higher than a control subject. For example, a subject having a cardiovascular disease or is at risk of developing a cardiovascular disease may have a CRP level that is at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold higher than a control subject. In some embodiments, a subject having a cardiovascular disease or is at risk of developing a cardiovascular disease has a CRP level that is 20%, 30%, 40%, 50%, 60%, 70%, 80%, 90%, 100%, 2-fold, 5-fold, 10-fold, 100-fold, or 1000-fold higher than a control subject. In some embodiments, a control subject is a healthy subject.

“Reduce the level or activity of CRP” means that the level or activity of CRP is reduced by at least 20% in the subject received treatment with the lipid lowering agent and the anti-inflammatory agent described herein, relative to before receiving the treatment. For example, the level or activity of CRP may be reduced by at least 20%, at least 30%, at least 40%, at least 50%, at least 60%, at least 70%, at least 80%, at least 90%, at least 95%, or 100% in the subject received treatment with the lipid lowering agent and the anti-inflammatory agent described herein, relative to before receiving the treatment. In some embodiments, the level or activity of CRP is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment.

In some embodiments, the level or activity of one or more lipids (e.g., one or more of non-HDL-C, LDL-C, VLDL-C, total cholesterol, and triglyceride) is reduced in the subject received treatment with the lipid lowering agent and the anti-inflammatory agent described herein, relative to before receiving the treatment. “Reduce the level or activity of one or more lipids” means that the level or activity of the one or more lipids (e.g., one or more of non-HDL-C, LDL-C, VLDL-C, total cholesterol, and triglyceride) is reduced by at least 20% lower when the composition is administered to the subject, compared to without the composition. For example, the level or activity of the one or more lipids (e.g., one or more of non-HDL-C, LDL-C, VLDL-C, total cholesterol, and triglyceride) may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the level or activity of the one or more lipids (e.g., one or more of non-HDL-C, LDL-C, VLDL-C, total cholesterol, and triglyceride) is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment. One skilled in the art can assess the activity of a lipid using routine methods.

In some embodiments, the level or activity of Apolipoprotein B (ApoB) is reduced in the subject received treatment with the lipid lowering agent and the anti-inflammatory agent described herein, relative to before receiving the treatment. “Reduce the level or activity of Apolipoprotein B (ApoB)” means that the level or activity of ApoB is reduced by at least 20% lower when the composition is administered to the subject, compared to without the composition. For example, the level or activity of ApoB may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the level or activity of ApoB is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment. One skilled in the art can assess the activity of ApoB using routine methods, e.g., immunostaining or western blotting.

In some embodiments, the ratio of total cholesterol to HDL-C is reduced in the subject received treatment with the lipid lowering agent and the anti-inflammatory agent described herein, relative to before receiving the treatment. “Reduce the ratio of total cholesterol to HDL-C” means that the ratio of total cholesterol to HDL-C is reduced by at least 20% lower when the composition is administered to the subject, compared to without the composition. For example, the ratio of total cholesterol to HDL-C may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the ratio of total cholesterol to HDL-C is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment.

In some embodiments, the occurrence of non-fatal myocardial infarction and/or cardiovascular mortality is reduced in the subject received treatment with the lipid lowering agent and the anti-inflammatory agent described herein, relative to before receiving the treatment. “Reduce the occurrence of non-fatal myocardial infarction and/or cardiovascular mortality” means that the occurrence of non-fatal myocardial infarction and/or cardiovascular mortality is reduced by at least 20% lower when the composition is administered to the subject, compared to without the composition. For example, the occurrence of non-fatal myocardial infarction and/or cardiovascular mortality may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the occurrence of non-fatal myocardial infarction and/or cardiovascular mortality is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment.

In some embodiments, the occurrence of non-fatal stroke is reduced in the subject received treatment with the lipid lowering agent and the anti-inflammatory agent described herein, relative to before receiving the treatment. “Reduce the occurrence of non-fatal stroke” means that the occurrence of non-fatal stroke is reduced by at least 20% lower when the composition is administered to the subject, compared to without the composition. For example, the occurrence of non-fatal stroke may be reduced by at least 20%, at least 30%, at least 40%, at least 50%, at least 60%, at least 70%, at least 80%, at least 90%, at least 95%, or 100% lower in the subject received treatment with the lipid lowering agent and the anti-inflammatory agent described herein, relative to before receiving the treatment. In some embodiments, the occurrence of non-fatal stroke is reduced by 20%, 30%, 40%, 50%, 60%, 70%, 80%, 90%, 95%, or 100% in the subject received treatment with the lipid lowering agent and the anti-inflammatory agent described herein, relative to before receiving the treatment.

In some embodiments, the lipid lowering agent and the anti-inflammatory agent are administered together (e.g., in the same composition). In some embodiments, the lipid lowering agent and the anti-inflammatory agent are administered separately (e.g., sequentially). For example, in some embodiments, the lipid lowering agent is administered first and the anti-inflammatory agent is administered second. In some embodiments, the anti-inflammatory agent is administered first and the lipid lowering agent is administered second.

In some embodiments, the lipid lowering agent and/or the anti-inflammatory agent is formulated in one or more compositions for administration to the subject. In some embodiments, the composition is a pharmaceutical composition. In some embodiments, the composition further comprises a pharmaceutically acceptable carrier. The pharmaceutical composition can further comprise additional agents (e.g. for specific delivery, increasing half-life, or other therapeutic agents). The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the composition comprising an anti-inflammatory agent from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the composition comprising an anti-inflammatory agent of the present disclosure in a composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the composition comprising an anti-inflammatory agent of the disclosure does not absorb are used.

In other embodiments, the composition comprising a lipid lowering agent and/or an anti-inflammatory agent is delivered in a controlled release system. In one embodiment, a pump may be used (see, e.g., Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a pharmaceutical composition adapted for intravenous or subcutaneous administration to a subject, e.g., a human being. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. The composition comprising a lipid lowering agent and/or an anti-inflammatory agent can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757.

The pharmaceutical compositions of the present disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, the pharmaceutical composition can be provided as a pharmaceutical kit comprising (a) a container containing a composition comprising an anti-inflammatory agent of the disclosure in lyophilized form and (b) a second container containing a pharmaceutically acceptable diluent (e.g., sterile water) for injection. The pharmaceutically acceptable diluent can be used for reconstitution or dilution of the lyophilized composition of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In some embodiments, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a lipid lowering agent and/or an anti-inflammatory agent. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Other aspects of the present disclosure provide methods of predicting a recurrence rate of a cardiovascular disease in a subject who has received or is undergoing therapy with the lipid lowering agent, the method comprising measuring a level of C-reactive protein (CRP) in the subject and determining that the subject is likely to have recurrence of the cardiovascular disease if the CRP level is above a pre-determined value.

In some embodiments, the subject (e.g., human subject) already has had a primary (first) cardiovascular event, such as, for example, a myocardial infarct or has had an angioplasty. A subject (e.g., human subject) who has had a primary cardiovascular event is at an elevated risk of a secondary (second) cardiovascular event. In some embodiments, the subject (e.g., human subject) has not had a primary cardiovascular event, but is at risk of having a cardiovascular event because the subject (e.g., human subject) has one or more risk factors to have a cardiovascular event. Examples of risk factors for a primary cardiovascular event include: hyperlipidemia, obesity, diabetes mellitus, hypertension, pre-hypertension, elevated level(s) of a marker of systemic inflammation, age, a family history of cardiovascular events, and cigarette smoking. The degree of risk of a cardiovascular event depends on the multitude and the severity or the magnitude of the risk factors that the subject (e.g., human subject) has. Risk charts and prediction algorithms are available for assessing the risk of cardiovascular events in a subject (e.g., human subject) based on the presence and severity of risk factors. One such example is the Framingham Heart Study risk prediction score. The subject (e.g., human subject) is at an elevated risk of having a cardiovascular event if the subject's 10-year calculated Framingham Heart Study risk score is greater than 5%, 6%, 7%, 8%, 9%, 10%, 11%, 12%, 13%, 14%, 15%, 16%, 17%, 18%, 19%, or 20%. In some embodiments, the subject who has or is at risk of developing a cardiovascular disease has an elevated CRP level, compared to a healthy subject. Other methods of assessing the risk of a cardiovascular event in a subject (e.g., human subject) include coronary calcium scanning, cardiac magnetic resonance imaging, and/or magnetic resonance angiography.

“Recurrence rate of a cardiovascular disease” refers to the likelihood of the subject experiencing a future cardiovascular after receiving therapy with a lipid lowering agent (e.g., statin and/or PCSK9 inhibitor). In some embodiments, the subject has been diagnosed of a cardiovascular disease and has received therapy or is undergoing therapy with a lipid lowering agent. In some embodiments, the subject has been diagnosed of being at risk of developing a cardiovascular disease and has received therapy or is undergoing therapy with a lipid lowering agent. In some embodiments, the subject is also receiving other therapeutic agents to treat or to reduce the risk of a cardiovascular event (e.g., any of the therapeutic methods described herein). In some embodiments, the therapy also can be non-drug treatments such as diet and/or exercise.

In some embodiments, the subject received the therapy with a lipid lowering agent for at least 2 weeks, at least 3 weeks, at least 4 weeks, at least 2 months, at least 3 months, at least 4 months, at least 5 months, at least 6 months or longer.

A “predetermined value” can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quartiles, the lowest quartile being individuals with the lowest risk and the highest quartile being individuals with the highest risk, or into tertiles the lowest tertile being individuals with the lowest risk and the highest tertile being individuals with the highest risk.

The predetermined value can depend upon the particular population of subject (e.g., human subject) selected. For example, an apparently healthy population will have a different ‘normal’ range of markers of systemic inflammation than will as a population the subject (e.g., human subject) of which have had a prior cardiovascular event. Accordingly, the predetermined values selected may take into account the category in which a subject (e.g., human subject) falls. Appropriate ranges and categories can be selected with no more than routine experimentation by those of ordinary skill in the art.

In some embodiments, the method further comprises measuring the level of a lipid such as, for example, a level of cholesterol or a level of a cholesterol fraction such as LDLC for characterizing a subject (e.g., human subject)'s risk of developing a future cardiovascular event. A level of a marker of systemic inflammation in the subject (e.g., human subject) is obtained. The level of the marker is compared to a predetermined value to establish a first risk value. A level of lipid in the subject (e.g., human subject) also is obtained. The level of the lipid in the subject (e.g., human subject) is compared to a second predetermined value to establish a second risk value. The subject (e.g., human subject)'s risk profile of developing the cardiovascular event then is characterized based upon the combination of the first risk value and the second risk value, wherein the combination of the first risk value and second risk value establishes a third risk value different from the first and second risk values. In some embodiments, the third risk value is greater than either of the first and second risk values. The cardiovascular event can be any cardiovascular event such as described above.

As is known in the art, cholesterol is an important normal body constituent, used in the structure of cell membranes, synthesis of bile acids, and synthesis of steroid hormones. Since cholesterol is water insoluble, most serum cholesterol is carried by lipoproteins (chylomicrons, VLDL-C, LDL-C, and HDL-C). Excess cholesterol in the blood has been correlated with cardiovascular events. LDL is sometimes referred to as “bad” cholesterol, because elevated levels of LDL correlate most directly with cardiovascular events such as coronary heart disease. HDL is sometimes referred to as “good” cholesterol since high levels of HDL are correlated with a reduced risk for cardiovascular events such as coronary heart disease. The term cholesterol means “total” cholesterol i.e. VLDL-C+LDL-C+HDL-C cholesterol.

In some embodiments, cholesterol levels are measured after a patient receives treatment with lipid lowering agents. The cholesterol measurement is typically reported in milligrams per deciliter (mg/dL). Typically, the higher the total cholesterol, the more at risk a subject (e.g., human subject) is for a cardiovascular event. A value of total cholesterol of less than 200 mg/dL is a “desirable” level and places the subject (e.g., human subject) in a group at less risk for a cardiovascular event(s). Levels over 240 mg/dL, for example, may put a subject (e.g., human subject) at almost twice the risk of cardiovascular event such as coronary heart disease as compared to someone with a level less than 200 mg/dL.

In some embodiments, LDL-C level is one of the predictors of risk of cardiovascular event. Typically, the higher the LDLC, the more at risk a subject (e.g., human subject) is for cardiovascular event. Levels of LDLC over 160 mg/dL may put a subject (e.g., human subject) at higher risks of a cardiovascular event(s) as compared to someone with a level less than 160 mg/dL. Levels of LDLC over 130 mg/dL in subject (e.g., human subject) with one or more risk factors for a future cardiovascular event may put a subject (e.g., human subject) at higher risks of a cardiovascular event(s) as compared to someone with a level less than 130 mg/dL. A level of LDLC less than 100 mg/dL is desirable in a subject (e.g., human subject) who has had a prior cardiovascular event and is on therapy to reduce the risk of a future cardiovascular event and places the subject (e.g., human subject) in a group at less risk for a cardiovascular event. A level of LDL-C less than 70 mg/dL is even more desirable in a such a subject (e.g., human subject) to reduce the risk of a future cardiovascular event.

In some embodiments, the subject who has received or is undergoing therapy with a lipid lowering agent has a healthy lipid (e.g., LDL-C or total cholesterol) level. In some embodiments, the subject who has received or is undergoing therapy with a lipid lowering agent has a healthy lipid (e.g., LDL-C or total cholesterol) level. As described herein, a subject who has received or is undergoing therapy with a lipid lowering agent and has a healthy lipid level may still be at risk of re-experiencing a cardiovascular event (i.e., has high recurrence rate of a cardiovascular disease) if the subject has a CRP level that is above a predetermined value. The subject may be determined to have a low recurrence rate of a cardiovascular disease if both the lipid level and the CRP level are below a predetermined, healthy level.

CRP level in the subject can be determined by a CRP blood test(s). Tests and methods for measuring CRP levels in blood, especially serum samples, and for interpreting results of such tests are widely used in clinical practice today. Since CRP is an acute phase protein that is synthesized in the liver and released into the blood stream in during inflammation, it's levels may be low in a subject without severe inflammation (e.g., inflammation caused by infection). Thus, in some embodiments, to assess a risk for a cardiovascular disease, the CRP level is measured by highly sensitive methods (hsCRP) that are capable detecting low levels of CRP (e.g., that in a healthy subject).

In some embodiments, the predetermined value of CRP level is about 3 mg/L of blood (i.e., blood sample from the subject (e.g., human subject)). In some embodiments, the predetermined value of CRP level is about 2 mg/L of blood. In some embodiments, the predetermined value of CRP level is about 1.75 mg/L of blood. In some embodiments, the predetermined value of CRP level is about 1.50 mg/L of blood. In some embodiments, the predetermined value of CRP level is about 1.25 mg/L of blood. In some embodiments, the predetermined value of CRP level is about 1 mg/L of blood. When ranges are employed, in some embodiments, the predetermined value of CRP level is below about 1-3 mg/L (e.g., 1-3, 2-3, 1-3 mg/L) of blood and another of the ranges is above about 3 mg/L of blood.

Subjects that have received or are undergoing a therapy with a lipid-lowering agent is determined to have high recurrence rate of a cardiovascular event if the subject has a CRP level of above the predetermined level. Other aspects of the present disclosure provide methods of reducing a recurrence rate of a cardiovascular disease in a subject who has received or is undergoing therapy with a lipid lowering agent, the method comprising administering to the subject an effective amount of an anti-inflammatory agent.

The terms “treatment” or “to treat” refer to both therapeutic and prophylactic treatments. If the subject is in need of treatment of a cardiovascular disease, then “treating the condition” refers to ameliorating, reducing or eliminating one or more symptoms associated with the cardiovascular disease or the severity of a cardiovascular disease or preventing any further progression of a cardiovascular disease. If the subject in need of treatment is one who is at risk of having a cardiovascular disease, then treating the subject refers to reducing the risk of the subject having a cardiovascular disease or preventing the subject from developing a cardiovascular disease.

A “subject” shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof. A subject in need thereof can be a subject who has or is at risk of developing a cardiovascular disease.

The agents (e.g., anti-inflammatory agents, lipid-reducing agents, and/or bispecific antibodies) described herein may be formulated in pharmaceutical compositions for administration to a subject. Pharmaceutically compositions that may be used in accordance with the present disclosure may be directly administered to the subject or may be administered to a subject in need thereof in a therapeutically effective amount. The term “therapeutically effective amount” refers to the amount necessary or sufficient to realize a desired biologic effect. For example, a therapeutically effective amount of a composition comprising a lipid lowering agent and/or an anti-inflammatory agent associated with the present disclosure may be that amount sufficient to ameliorate one or more symptoms of the disease or disorder. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular pharmaceutically compositions being administered the size of the subject, or the severity of the disease or condition. One of ordinary skill in the art can empirically determine the effective amount of a particular therapeutic compound associated with the present disclosure without necessitating undue experimentation.

Subject doses of the composition comprising a lipid lowering agent and/or an anti-inflammatory agent described herein for delivery typically range from about 0.1 μg to 10 mg per administration, which depending on the application could be given daily, weekly, or monthly and any other amount of time there between. In some embodiments, a single dose is administered during the critical consolidation or reconsolidation period. The doses for these purposes may range from about 10 μg to 5 mg per administration, and most typically from about 100 μg to 1 mg, with 2-4 administrations being spaced, for example, days or weeks apart, or more. In some embodiments, however, parenteral doses for these purposes may be used in a range of 5 to 10,000 times higher than the typical doses described above.

In some embodiments, a composition comprising a lipid lowering agent and/or an anti-inflammatory agent, or a bispecific antibody described herein is administered at a dosage of between about 1 and 10 mg/kg of body weight of the mammal. In other embodiments, a composition comprising a lipid lowering agent and/or an anti-inflammatory agent, or a bispecific antibody described herein is administered at a dosage of between about 0.001 and 1 mg/kg of body weight of the mammal. In yet other embodiments, a composition comprising a lipid lowering agent and/or an anti-inflammatory agent, or a bispecific antibody described herein is administered at a dosage of between about 10-100 ng/kg, 100-500 ng/kg, 500 ng/kg-1 mg/kg, or 1-5 mg/kg of body weight of the mammal, or any individual dosage therein.

The formulations of the present disclosure are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the composition comprising a lipid lowering agent and/or an anti-inflammatory agent, or a bispecific antibody described herein can be administered to a subject by any mode that delivers the composition to the desired location, e.g., mucosal, injection, systemic, etc. Administering the pharmaceutical composition of the present disclosure may be accomplished by any means known to the skilled artisan. In some embodiments, the composition comprising an anti-inflammatory agent and/or an anti-inflammatory agent, or a bispecific antibody described herein is administered subcutaneously, intracutaneously, intravenously, intramuscularly, intraarticularly, intraarterially, intrasynovially, intrasternally, intrathecally, intralesionally, or intracranially.

For oral administration, the composition can be formulated readily by combining the active compound(s) with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

In some embodiments, the composition can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the lipid lowering agent and/or the anti-inflammatory agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the lipid lowering agent and/or the anti-inflammatory agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The pharmaceutical compositions of the present disclosure, when desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the composition may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The pharmaceutical compositions of the present disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The subjects of the present disclosure have or are at risk of developing a cardiovascular disease. A “cardiovascular disease (CVD)” is a class of diseases that involve the heart or blood vessels. Non-limiting examples of cardiovascular disease include: coronary artery diseases (CAD) such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, venous thrombosis, acute coronary syndrome, myocardial ischemia, chronic stable angina pectoris, unstable angina pectoris, coronary re-stenosis, coronary stent re-stenosis, coronary stent re-thrombosis, revascularization, angioplasty, transient ischemic attack, pulmonary embolism, vascular occlusion, and cardiovascular death.

Coronary artery disease (CAD), also known as ischemic heart disease (IHD), is a group of diseases that includes: stable angina, unstable angina, myocardial infarction, and sudden cardiac death. Risk factors for CAD include: high blood pressure, smoking, diabetes, lack of exercise, obesity, high blood cholesterol, poor diet, and excessive alcohol, and/or depression. The underlying mechanism involves reduction of blood flow and oxygen due to atherosclerosis of the arteries of the heart.

Myocardial infarction (MI), commonly known as a heart attack, occurs when blood flow decreases or stops to a part of the heart, causing damage to the heart muscle. Risk factors for MI include high blood pressure, smoking, diabetes, lack of exercise, obesity, high blood cholesterol, poor diet, and excessive alcohol intake.

Myocardial ischemia occurs when blood flow to your heart is reduced, preventing it from receiving enough oxygen. The reduced blood flow is usually the result of a partial or complete blockage of your heart's arteries (coronary arteries).

Angina pectoris is the medical term for chest pain or discomfort due to coronary heart disease. It occurs when the heart muscle doesn't get as much blood as it needs. This usually happens because one or more of the heart's arteries is narrowed or blocked, also called ischemia. Unstable angina (UA) is a type of angina pectoris that is irregular.

Stroke is a medical condition in which poor blood flow to the brain results in cell death. There are two main types of stroke: ischemic, due to lack of blood flow, and hemorrhagic, due to bleeding. Risk factors for stroke include high blood pressure, smoking, obesity, high blood cholesterol, diabetes mellitus, previous TIA, and atrial fibrillation. Acute coronary syndrome is a term used to describe a range of conditions associated with sudden, reduced blood flow to the heart. A transient ischemic attack (TIA) is like a stroke, producing similar symptoms, but usually lasting only a few minutes and causing no permanent damage.

Heart failure (HF), often referred to as congestive heart failure, occurs when the heart is unable to pump sufficiently to maintain blood flow to meet the body's needs. Common causes of heart failure include coronary artery disease including a previous myocardial infarction (heart attack), high blood pressure, atrial fibrillation, valvular heart disease, excess alcohol use, infection, and cardiomyopathy of an unknown cause.

Rheumatic heart disease is a complication of rheumatic fever in which the heart valves are damaged. Rheumatic fever (RF) is an inflammatory disease that can involve the heart, joints, skin, and brain.

Cardiomyopathy is a group of diseases that affect the heart muscle. Types of cardiomyopathy include hypertrophic cardiomyopathy, dilated cardiomyopathy, restrictive cardiomyopathy, arrhythmogenic right ventricular dysplasia, and broken heart syndrome. Dilated cardiomyopathy may also result from alcohol, heavy metals, coronary heart disease, cocaine use, and viral infections. Restrictive cardiomyopathy may be caused by amyloidosis, hemochromatosis, and some cancer treatments.

Peripheral artery disease (PAD) is a narrowing of the arteries other than those that supply the heart or the brain. Risk factors for PAD include cigarette smoking, diabetes, high blood pressure, and high blood cholesterol. The underlying mechanism is usually atherosclerosis.

A congenital heart defect (CHD), also known as a congenital heart anomaly or congenital heart disease, is a problem in the structure of the heart that is present at birth. Valvular heart disease is any disease process involving one or more of the four valves of the heart (the aortic and mitral valves on the left and the pulmonary and tricuspid valves on the right). Carditis is the inflammation of the heart or its surroundings. An aortic aneurysm is an enlargement (dilation) of the aorta to greater than 1.5 times normal size.

Thrombosis is the formation of a blood clot inside a blood vessel, obstructing the flow of blood through the circulatory system. A venous thrombus is a blood clot (thrombus) that forms within a vein. Pulmonary embolism is the sudden blockage of a major blood vessel (artery) in the lung, usually by a blood clot. Vascular occlusion is a blockage of a blood vessel, usually with a clot. It differs from thrombosis in that it can be used to describe any form of blockage, not just one formed by a clot. When it occurs in a major vein, it can, in some cases, cause deep vein thrombosis.

Coronary re-stenosis is the recurrence of stenosis, a narrowing of a blood vessel, leading to restricted blood flow. Coronary stent re-stenosis occurs when a stent is implanted and restenosis is developing inside the stent. Coronary stent re-thrombosis occurs when a stent is implanted and thrombosis develops inside the stent.

Revascularization is the restoration of perfusion to a body part or organ that has suffered ischemia. It is typically accomplished by surgical means. Vascular bypass and angioplasty are the two primary means of revascularization.

The present disclosure is illustrated but not limited by reference to the following Examples.

### EXAMPLES

Patients with residual inflammatory risk have high rates of recurrent cardiovascular events due to persistently elevated levels of high sensitivity C-reactive protein (hsCRP) despite aggressive use of statin therapy.¹⁻⁷ Such patients, commonly defined as those taking statin therapy who have hsCRP≥2 mg/L and LDL cholesterol <70 mg/dl,⁸ comprise nearly 30 percent of patients in contemporary practice and are twice as common as those with residual cholesterol risk (defined by LDL levels ≥70 mg/dL and hsCRP<2 mg/L).⁹ Recently, the Canakinumab Anti-inflammatory Thrombosis Outcomes Study (CANTOS) demonstrated that IL-1β inhibition with canakinumab significantly reduces both hsCRP and cardiovascular events,¹⁰ data providing the first specific treatment for patients with residual cholesterol risk. Indeed, the magnitude of risk reduction in CANTOS was virtually identical to that achieved in the FOURIER and SPIRE proprotein convertase subtilisin-kexin type 9 (PCSK9) trials,^{11, 12} despite no change in LDL cholesterol. Importantly, the absolute event rates of 5.3% and 9.1% at 1-year and 2-years of follow-up in FOURIER inform us that many patients achieving very low LDL-C levels will continue to experience vascular events. Whether residual inflammatory risk remains an important clinical issue among statin treated patients who additionally receive PCSK9 inhibition is unknown. This issue was addressed in the recently completed SPIRE-1 and SPIRE 2 trials described herein.

### Methods

### Study Population and Procedures

The SPIRE bococizumab development program consisted of two parts: the six SPIRE lipid-lowering studies and the SPIRE-1 and SPIRE-2 event-driven cardiovascular trials. The design and primary findings of SPIRE-1 and SPIRE-2 have been previously published.^{12, 13} The virtually identical designs of the two trials permitted them to be combined according to an integrated statistical analysis plan. In brief, patients were eligible for enrollment if they had either a prior cardiovascular event (secondary prevention cohort) or a history of diabetes, chronic kidney disease, or peripheral vascular disease with additional cardiovascular risk conditions or a history of familial hypercholesterolemia (high-risk primary prevention cohort). All patients were required to have received at least 4 weeks of stable statin therapy (atorvastatin 40 mg/day, rosuvastatin 20 mg/day, or simvastatin 40 mg/d) unless they could not take those doses without side effects and were thus on lower intensity statin therapy or had complete statin intolerance (eligible for SPIRE-2 only). Patients were required to have a directly measured LDL-C level of at least 70 mg/dL in SPIRE-1 and of >100 mg/dL in SPIRE-2. Patients were also eligible according to their non-HDL cholesterol level at entry (100 mg/dL for SPIRE-1 and 130 mg/dL for SPIRE-2). In a double-blinded fashion, patients were randomized in a 1:1 ratio to treatment with subcutaneous bococizumab 140 mg every 2 weeks or matching placebo. The SPIRE program was sponsored by Pfizer.

The study population for the current analysis comprises the subgroup of patients who were receiving statin therapy, were allocated to active bococizumab and had available baseline and 14 week hsCRP available for analysis (n=9,738). All patients provided written informed consent. Ethics committees at each center approved the protocol.

### Endpoints

The pre-specified primary endpoint of the two trials was a composite of adjudicated and confirmed nonfatal myocardial infarction, nonfatal stroke, hospitalization for unstable angina requiring urgent revascularization, or cardiovascular death. All incident events that were components of these endpoints were adjudicated by a committee in which the members were unaware of treatment assignments.

### Statistical Analyses

Of 13,675 patients randomized to the active treatment arm, 12,711 (93.0%) were receiving statin therapy, and 9,738 (71.2%) also had hsCRP_{OT} levels available at the 14 week timepoint. The corresponding proportion of patients randomized to placebo, receiving statin therapy and having follow-up biomarker levels was 9,785 (71.6%).

The study population was then restricted to individuals allocated to bococizumab and divided into three groups according to hsCRP_{OT} level <1, 1-3, and >3 mg/dL comprising 30.4%, 34.8%, and 34.9% of patients, respectively. When cut points of <2 and ≥2 mg/dl were used, these percentages were 52.8% and 47.2%. Baseline characteristics according the three primary hsCRP_{OT} groups were summarized using percentages for categorical values and medians (interquartile ranges) for continuous variables. Trends in these characteristics across ordered hsCRP_{OT} categories were assessed using the Cochran-Armitage trend test for differences in proportions and the Jonckheere-Terpstra test for differences in medians.

To evaluate the treatment effect of bococizumab on lipid levels and on hsCRP, median on-treatment levels were determined at baseline and 14 weeks of therapy. Linear mixed model repeated measure analysis conditioning on the baseline value were constructed with the independent value being the biomarker of interest using log transformation as deemed appropriate for non-normal distributions. The mean percent change and bococizumab treatment effect was estimated by fitting terms corresponding to the study drug assignment. Percent change in lipid levels in each hsCRP_{OT} group among patients allocated to bococizumab was then estimated using mixed models as before, conditioning on the baseline value and fitting a term corresponding to the hsCRP_{OT} group.

Cox proportional hazards models were used to estimate hazard ratios (HRs) according to hsCRP_{OT} group. Three adjusted models are presented which adjusted for: 1) age and sex, 2) age, sex, traditional cardiovascular risk factors (including current smoking, diabetes, hypertension, and body-mass index) plus statin intensity at enrollment (moderate-intensity or high-intensity), and 3) model 2 variables and plus on-treatment LDL-C (LDL_{OT}). For each model, a test for trend across hsCRP_{OT} categories was performed after assigning the median value to each group. All analyses were stratified by study (SPIRE-1 or SPIRE-2), region, and screening LDL-C threshold (<70 or <100 mg/dL). Further tests assessed for heterogeneity in treatment effects of bococizumab versus placebo according to hsCRP_{OT} groups by use of an interaction term (bococizumab x hsCRP_{OT} group).

To permit comparison to associations for on-treatment LDL-C measured at 14 weeks, the study population was additionally divided into LDL_{OT} groups (approximate tertiles) using the categories of <30, 30-50, and >50 mg/dL and comparable Cox models used to estimated adjusted HRs in each of these groups. Cutpoints of < or ≥2 mg/L for hsCRP and < or ≥40 mg/dl for LDL-C were also used. Finally, to examine the risk association throughout the range of hsCRP_{OT}, the relationship between hsCRP_{OT} and cardiovascular event rates was plotted using a smoothing function to the average of estimated event rates at each hsCRP_{OT} level based on adjusted Cox models.

### Results

Study Population by On-Treatment hsCRP Levels

The study population comprised 2958 (30.4%) with hsCRP_{OT}<1 mg/L, 3385 (34.8%) with hsCRP_{OT} 1-3 mg/L, and 3395 (34.9%) with hsCRP_{OT}>3 mg/L. Baseline characteristics according to hsCRP_{OT} are shown in Table 1. Patients with higher hsCRP_{OT} groups were more likely to be women, to be obese, have diabetes or diagnosed hypertension, and to be current smokers but less likely to have prior cardiovascular disease. Several baseline lipid parameters were also significantly different across increasing hsCRP group, including higher levels of LDL-C, total cholesterol (TC), non-HDL cholesterol (non-HDL-C), triglycerides, total:HDL-C ratio, and apolipoprotein B (apoB) and lower levels of HDL-C.

TABLE 1
Baseline Characteristics According to hsCRP_{OT} at 14 Weeks

| | hsCRP_{OT} Group | | | | | | |
|---|---|---|---|---|---|---|---|
| | <1 mg/L | | 1-3 mg/L | | >3 mg/L | | |
| | N = 2958 | | N = 3385 | | N = 3395 | | |
| Baseline Characteristic | (30.4%) | | (34.8%) | | (34.9%) | | P-value |
| Age, years | 63 | (56, 69) | 64 | (57, 70) | 63 | (57, 69) | <0.19 |
| Female Sex, % | 28.0 | | 29.4 | | 31.4 | | <0.001 |
| Body-Mass Index, kg/m² | 27.9 | (25.5, 30.9) | 29.4 | (26.6, 32.7) | 31.4 | (27.9, 35.9) | <0.001 |
| Diabetes, % | 37.4 | | 47.2 | | 60.2 | | <0.001 |
| Hypertension, % | 75.1 | | 82.4 | | 87.4 | | <0.001 |
| Current Smoking, % | 19.0 | | 23.8 | | 30.0 | | <0.001 |
| High-Risk Primary | 8.1 | | 14.0 | | 19.2 | | <0.001 |
| Prevention, % | | | | | | | |
| US/Canada, % | 21.3 | | 27.4 | | 35.6 | | <0.001 |
| Statin Regimen, % | | | | | | | |
| Moderate Intensity | 8.3 | | 9.0 | | 9.4 | | <0.10 |
| High Intensity | 91.8 | | 91.0 | | 90.6 | | |
| LDL Cholesterol, mg/dL | 92.4 | (80.5, 110.4) | 96.5 | (82.4, 118.0) | 101.0 | (85.1, 125.5) | <0.001 |
| Total Cholesterol, mg/dL | 161.8 | (144.8, 184.2) | 166.2 | (147.5, 193.1) | 171.5 | (151.2, 200.5) | <0.001 |
| Non-HDL Cholesterol, | 112.0 | (97.1, 132.2) | 117.7 | (101.2, 143.8) | 124.9 | (105.5, 153.9) | <0.001 |
| mg/dL | | | | | | | |
| HDL Cholesterol, mg/dL | 47.0 | (40.0, 56.0) | 45.2 | (38.4, 53.5) | 43.8 | (37.0, 52.5) | <0.001 |
| Triglycerides, mg/dL | 116.5 | (87.6, 160.2) | 137.5 | (101.3, 192.5) | 149.6 | (108.0, 210.6) | <0.001 |
| Total:HDL Cholesterol Ratio | 3.4 | (2.9, 4.1) | 3.7 | (3.1, 4.4) | 3.9 | (3.2, 4.7) | <0.001 |
| Apolipoprotein B, mg/dL | 78 | (68, 91) | 82 | (71, 98) | 87 | (74, 105) | <0.001 |
| High-Sensitivity CRP, mg/L | 0.7 | (0.4, 1.2) | 1.8 | (1.1, 2.9) | 4.7 | (2.7, 7.6) | <0.001 |

Percentages may not add up to 100% due to rounding.

Bococizumab Treatment Effects on Lipid Levels, hsCRP, and Cardiovascular Events

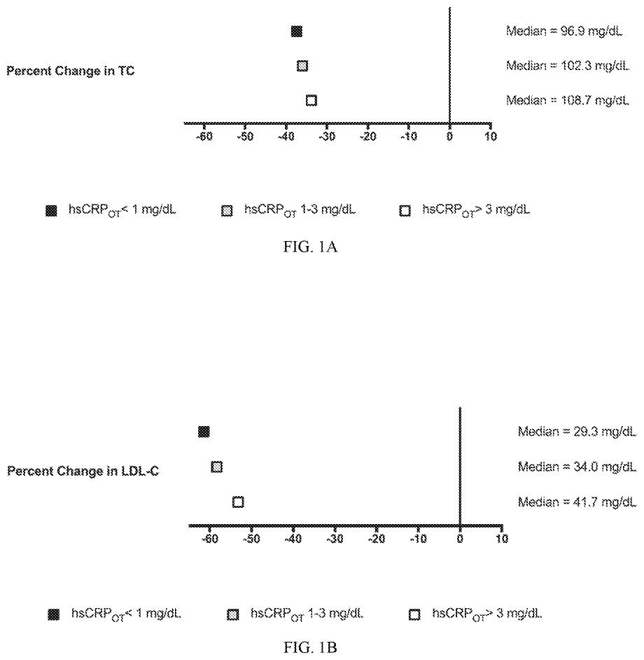

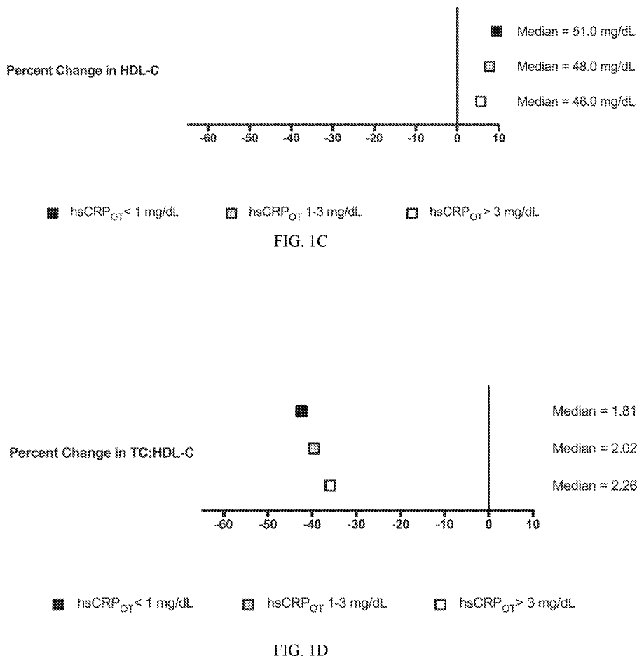

When compared to placebo, bococizumab was associated with statistically significant reductions in LDL-C (−60.5%), TC (−37.6%), non-HDL-C (−54.9%), TC:HDL-C ratio (−41.1%), apoB (−56.0%), and triglycerides (−19.9%) as well as an increase in HDL-C (+6.4%) (Table 2; all p<0.001). By contrast, there was no significant effect on hsCRP: mean percent change +6.6% (95% CI: −1.0 to 14.1; p=0.09; median change 0.0%) at 14 weeks and +6.7% (−9.3 to 16.9%; p=0.57; median change 0.0%) at 52 weeks (n=3267). Percent changes in lipid fractions were somewhat lower in magnitude in higher hsCRP_{OT} groups (FIG. 1). Nonetheless, even among those with hsCRP>3 mg/L, the median LDL-C_{OT} at 14 weeks was 41.7 (IQR 25.9, 67.0) mg/L. Bococizumab treatment effects by hsCRP_{OT} were similar in magnitude and there was no evidence of heterogeneity across hsCRP_{OT} groups (p-interaction=0.87).

TABLE 2
Median Lipid Levels and hsCRP at Baseline and 14 Weeks and Treatment Effect (Percent Change) with Bococizumab

| | Bococizumab | | | Placebo | | | Treatment Effect^{†} | | |
|---|---|---|---|---|---|---|---|---|---|
| Parameter | No. | Median | | No. | Median | | % Change | 95% CI | P-Value |
| LDL-C (mg/dL) | | | | | | | | | |
| Baseline | 9662 | 96.5 | (82.5, 118.0) | 9716 | 96.5 | (82.6, 117.5) | −60.5 | (−61.2 to −59.8) | <0.001 |
| 14 Weeks | 9662 | 34.7 | (22.4, 56.4) | 9716 | 97.7 | (82.0, 120.3) | | | |
| Total Cholesterol | | | | | | | | | |
| (mg/dL) | | | | | | | | | |
| Baseline | 9670 | 166.5 | (147.9, 192.3) | 9711 | 166.4 | (148.0, 191.9) | −37.6 | (−38.1 to −37.1) | <0.001 |
| 14 Weeks | 9670 | 102.7 | (84.2, 128.2) | 9711 | 167.6 | (146.7, 195.0) | | | |
| Non-HDL Cholesterol | | | | | | | | | |
| (mg/L) | | | | | | | | | |
| Baseline | 9648 | 118.0 | (101.0, 143.8) | 9690 | 117.8 | (101.4, 142.7) | −54.9 | (−55.1 to −53.7) | <0.001 |
| 14 Weeks | 9648 | 50.0 | (34.7, 76.0) | 9690 | 118.9 | (100.0, 146.3) | | | |
| HDL Cholesterol | | | | | | | | | |
| (mg/dL) | | | | | | | | | |
| Baseline | 9649 | 45.2 | (38.2, 54.0) | 9694 | 45.6 | (38.6, 54.3) | 6.4 | (6.1 to 6.8) | <0.001 |
| 14 Weeks | 9649 | 48.0 | (40.9, 57.9) | 9694 | 45.9 | (38.6, 54.8) | | | |
| Triglycerides | | | | | | | | | |
| (mg/dL) | | | | | | | | | |
| Baseline | 9699 | 134.5 | (98.2, 189.0) | 9713 | 133.6 | (98.5, 187.2) | −19.9 | (−21.0 to −18.8) | <0.001 |
| 14 Weeks | 9699 | 107.0 | (76.1, 157.5) | 9713 | 133.0 | (96.0, 189.4) | | | |
| Total:HDL | | | | | | | | | |
| Cholesterol Ratio | | | | | | | | | |
| Baseline | 9648 | 3.6 | (3.1, 4.4) | 9690 | 3.6 | (2.1, 4.4) | −41.1 | (−41.7 to −40.6) | <0.001 |
| 14 Weeks | 9648 | 2.0 | (1.7, 2.7) | 9690 | 3.6 | (3.0, 4.5) | | | |
| Apolipoprotein B | | | | | | | | | |
| (mg/dL) | | | | | | | | | |
| Baseline | 9641 | 82.0 | (71.0, 99.0) | 9678 | 82.0 | (71.0, 98.0) | −56.0 | (−56.7 to −55.2) | <0.001 |
| 14 Weeks | 9733 | 37.0 | (17.5, 56.0) | 9782 | 82.5 | (71.0, 99.0) | | | |
| hsCRP (mg/L) | | | | | | | | | |
| Baseline | 9738 | 1.88 | (0.87, 4.21) | 9756 | 1.90 | (0.85, 4.08) | 6.6 | (−1.0 to 14.1) | 0.09 |
| 14 Weeks | 9738 | 1.84 | (0.83, 4.19) | 9785 | 1.68 | (0.78, 3.88) | | | |

^{†}The percent change is from baseline to 14 weeks for the bococizumab group as compared with the placebo group.

Event Rates According to On-Treatment hsCRP and On-Treatment LDL

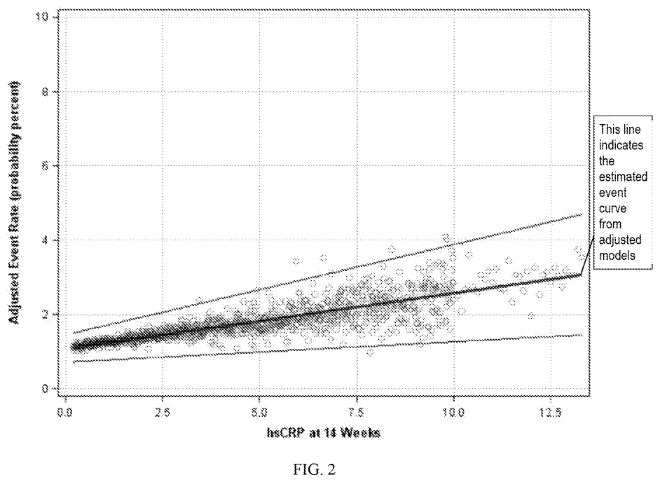

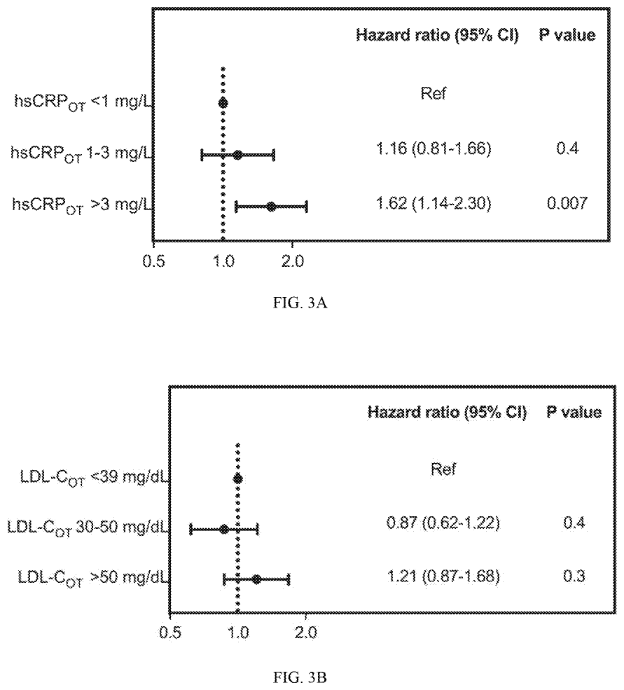

Overall, a monotonic increase in adjusted event probabilities for the primary CVD endpoint was observed with increasing on-treatment hsCRP levels (FIG. 2). Event rates in hsCRP_{OT} groups were 1.96, 2.50, and 3.59 per 100 person-years for hsCRP<1, 1-2, and >3 mg/L, respectively (Table 3). In multivariable models that adjusted for age and sex, the corresponding HRs for CVD were 1.0 (ref), 1.23 (95% CI 0.86 to 1.75) and 1.79 (95% CI 1.28 to 2.50); p-trend<0.001. In models additionally adjusting for traditional cardiovascular risk factors and baseline intensity of statin therapy, the HR comparing highest to lowest hsCRP_{OT} category (>3 vs. <1 mg/dL) was 1.67 (95% CI 1.18 to 2.37; p=0.02). Further adjustment for LDL_{OT} minimally attenuated this risk (model 3, Table 1 and FIG. 3A). In models additionally adjusting for on-treatment TC:HDL-C ratio, adjusted HRs were 1.0 (ref), 1.13, and 1.58 (p-trend=0.002). When individual components of the composite endpoint were examined, hsCRP_{OT} category was significantly with non-fatal myocardial infarction (adjusted HRs 1.0, 0.91, 1.46, p-trend=0.017), cardiovascular mortality (adjusted HRs 1.0, 1.60, 3.76, p-trend=0.002), and total mortality (adjusted HRs 1.0, 1.58, 3.45, p-trend<0.001). Similar but non-significant trends were noted for stroke and unstable angina requiring urgent coronary revascularization.

In parallel analyses in which patients were categorized according to LDL-C_{OT} (<30, 30-50, >50 mg/dl), the HRs for the primary CVD endpoint were 1.0 (ref), 0.87 (95% CI 0.62 to 1.22) and 1.21 (0.87 to 1.68) with p-trend=0.16 in analyses adjusting for model 3 covariates and hsCRP_{OT} instead of LDL-C_{OT} (FIG. 3B and Table 4). Similar findings were observed when the alternate cut points of ≥2 mg/L for hsCRP_{OT} and ≥40 mg/dl for LDL-C_{OT} were used (Tables 5 and 6).

TABLE 3
Hazard Ratios for the Cardiovascular Events^{†} According to hsCRP_{OT} at 14 weeks

| | hsCRP_{OT} Group | | | |
|---|---|---|---|---|
| | <1 mg/L | 1-3 mg/L | >3 mg/L | |
| | N = 2958 | N = 3385 | N = 3395 | |
| | (30.4%) | (34.8%) | (34.9) | |
| Primary Endpoint* | 52 | 76 | 109 | P-trend |
| Events per 100 person-years | 1.96 | 2.50 | 3.59 | |
| Model 1 | 1 (ref) | 1.23 (0.86 to 1.75) | 1.79 (1.28 to 2.50) | <0.001 |
| | | p = 0.3 | p = 0.001 | |
| Model 2 | 1 (ref) | 1.17 (0.82 to 1.68) | 1.67 (1.18 to 2.37) | <0.001 |
| | | p = 0.4 | p = 0.004 | |
| Model 3 | 1 (ref) | 1.16 (0.81 to 1.66) | 1.62 (1.14 to 2.30) | 0.001 |
| | | p = 0.4 | p = 0.007 | |
| Individual Endpoints (Model 3) | | | | |
| Nonfatal Myocardial | N = 31 | N = 36 | N = 61 | 0.017 |
| Infarction | 1 (ref) | 0.91 (0.56 to 1.49) | 1.46 (0.92 to 2.32) | |
| | | p = 0.7 | p = 0.11 | |
| Nonfatal Stroke | N = 7 | N = 14 | N = 14 | 0.4 |
| | 1 (ref) | 1.62 (0.65 to 4.05) | 1.47 (0.56 to 3.85) | |
| | | p = 0.3 | p = 0.4 | |
| Hospitalization for Unstable | N = 10 | N = 16 | N = 21 | 0.2 |
| Angina Requiring Urgent | 1 (ref) | 1.33 (0.60 to 2.95) | 1.65 (0.74 to 3.68) | |
| Revascularization | | p = 0.5 | P = 0.2 | |
| Cardiovascular Death | N = 5 | N = 11 | N = 23 | 0.002 |
| | 1 (ref) | 1.60 (0.54 to 4.73) | 3.76 (1.38 to 10.2) | |
| | | p = 0.4 | p = 0.009 | |
| Any Death | N = 10 | N = 20 | N = 38 | <0.001 |
| | 1 (ref) | 1.58 (0.73 to 3.41) | 3.45 (1.68 to 7.08) | |
| | | p = 0.3 | p = 0.001 | |

*The primary endpoint was nonfatal myocardial infarction, nonfatal stroke, hospitalization for unstable angina
Model 1: age- and sex-adjusted
Model 2: additionally adjusted for baseline smoking, diabetes, hypertension, body-mass index, baseline statin (moderate-, or high-intensity)
Model 3: additionally adjusted for 14 week on-treatment LDL-C
All models stratified by study (SPIRE-1 or SPIRE-2), region, and screening LDLc.

TABLE 4
Hazard Ratios for the Cardiovascular Events According to LDL-C_{OT} at 14 Weeks

| | LDL-C_{OT} Group | | | |
|---|---|---|---|---|
| | <30 mg/dL | 30-50 mg/dL | >50 mg/dL | |
| | N = 3979 | N = 2770 | N = 2913 | |
| | (41.2%) | (28.7%) | (30.1%) | |
| Primary Endpoint* | 88 | 57 | 90 | P-trend |
| Events per 100 person-years | 2.50 | 2.28 | 3.40 | |
| Model 1 | 1 (ref) | 0.90 (0.64 to 1.27) | 1.36 (0.98 to 1.87) | 0.04 |
| | | p = 0.6 | p = 0.07 | |
| Model 2 | 1 (ref) | 0.89 (0.63 to 1.25) | 1.28 (0.92 to 1.77) | 0.09 |
| | | p = 0.5 | p = 0.14 | |
| Model 3 | 1 (ref) | 0.87 (0.62 to 1.22) | 1.21 (0.87 to 1.68) | 0.16 |
| | | p = 0.4 | p = 0.3 | |

*The primary endpoint was nonfatal myocardial infarction, nonfatal stroke, hospitalization for unstable angina requiring urgent revascularization, or cardiovascular death.
*76 subjects excluded due to missing LDL-C_{OT}
Model 1: age- and sex-adjusted
Model 2: additionally adjusted for baseline smoking, diabetes, hypertension, body-mass index, baseline statin (moderate-, or high-intensity)
Model 3: additionally adjusted for on-treatment hsCRP_{OT}
All models stratified by study (SPIRE-1 or SPIRE-2), region, and screening LDL-C.

TABLE 5
Hazard Ratios for the Cardiovascular Events
According to hsCRP_{OT} at 14 weeks

hsCRP_{OT} Group

| | <2 mg/L | ≥2 mg/L |
|---|---|---|
| | N = 5143 | N = 4595 |
| | (52.8%) | (47.2%) |
| Primary Endpoint* | 104 | 133 |
| Events per 100 person-years | 2.25 | 3.23 |
| Model 1 | 1.0 (ref) | 1.40 (1.08 to 1.82) |
| | | p = 0.01 |
| Model 2 | 1.0 (ref) | 1.33 (1.01 to 1.74) |
| | | p = 0.04 |
| Model 3 | 1.0 (ref) | 1.29 (0.98 to 1.70) |
| | | p = 0.07 |

*The primary endpoint was nonfatal myocardial infarction, nonfatal stroke, hospitalization for unstable angina requiring urgent revascularization, or cardiovascular death.
Model 1: age- and sex-adjusted
Model 2: additionally adjusted for baseline smoking, diabetes, hypertension, body-mass index, baseline statin (moderate-, or high-intensity)
Model 3: additionally adjusted for on-treatment LDL-C_{OT} (no. missing = 76)
All models stratified by study (SPIRE-1 or SPIRE-2), region, and screening LDL-C.

TABLE 6
Hazard Ratios for the Cardiovascular Events
According to LDL-C_{OT} at 14 weeks

| | hsLDL-C_{OT} Group | |
|---|---|---|
| | <40 mg/dL | ≥40 mg/dL |
| | N = 5610 | N = 4052 |
| | (58.1%) | (41.9%) |
| Primary Endpoint* | 117 | 118 |
| Events per 100 person-years | 2.35 | 3.19 |
| Model 1 | 1.0 (ref) | 1.35 (1.03 to 1.78) |
| | | p = 0.03 |
| Model 2 | 1.0 (ref) | 1.29 (0.97 to 1.70) |
| | | p = 0.08 |
| Model 3 | 1.0 (ref) | 1.24 (0.93 to 1.64) |
| | | p = 0.14 |

*The primary endpoint was nonfatal myocardial infarction, nonfatal stroke, hospitalization for unstable angina requiring urgent revascularization, or cardiovascular death.
*76 subjects excluded due to missing LDL-C_{OT}
Model 1: age- and sex-adjusted
Model 2: additionally adjusted for baseline smoking, diabetes, hypertension, body-mass index, baseline statin (moderate-, or high-intensity)
Model 3: additionally adjusted for on-treatment hsCRP_{OT}
All models stratified by study (SPIRE-1 or SPIRE-2), region, and screening LDL-C.

### DISCUSSION

In this population of 9,738 high-risk patients concomitantly treated with statins and LDL-PSCK9 inhibition, 47.2% had residual inflammatory risk defined by on-treatment hsCRP level ≥2 mg/L, with 34.9% having values >3 mg/L. Individuals with persistent CRP elevation tended to be those with multiple risk factors including diabetes, obesity, hypertension, and mixed dyslipidemia, conditions known to correlate with, if not be driven by, a pro-inflammatory state. PCSK9 inhibition with bococizumab had no effect on hsCRP over time. Despite exceptionally aggressive reduction of LDL-C, there was a continuous gradient in risk for future vascular events according to on-treatment hsCRP. Compared to those without evidence of subclinical inflammation, those with on-treatment hsCRP>3 mg/L had a 62% increase in risk of future vascular events. Elevated hsCRP was significantly associated with increased rates of myocardial infarction, and cardiovascular death, and all-cause mortality.

There is broad consensus that atherosclerosis is both a disorder of lipid accumulation and inflammation. From a clinical perspective, extensive prior work has found hsCRP to be an independent predictor of cardiovascular events both in primary prevention and high-risk secondary prevention. Further, among patients with residual inflammatory risk, randomized clinical trials have proven the efficacy of statin therapy in primary prevention¹⁴ and anti-inflammatory therapy in secondary prevention¹⁰ It has been uncertain, however, whether residual inflammatory risk persists after the extremely aggressive reduction in LDL-C that can be achieved with the combination of statin therapy and PCSK9 inhibition. Importantly, in an era when ever more specialized therapies in cardiovascular medicine will continue to emerge, the call for biomarkers which inform clinicians about risk stratification, drug choice and dose, therapeutic responses, and ultimately personalized interventions will only be amplified.

In this context, these data have several important implications. First, these data clarify that PCSK9 inhibition has no effect on plasma measures of hsCRP despite large effects on atherogenic lipids. Second, the current data demonstrate that, despite inter-relationships of LDL oxidation and inflammation, the combination of high intensity statin therapy and PCSK9 inhibition does not fully address inflammatory mechanisms of atherothrombosis. In isolation, the post-hoc findings are associative and could still be explained by underlying conditions that promote subclinical inflammation. As such, we believe that combination therapy with PCSK9 inhibition and anti-inflammatory therapy will provide the optimal method to address residual cardiovascular risk. While canakinumab is currently the only anti-inflammatory agent proven to reduce cardiovascular events, clinical trials are currently in progress using colchicine and low-dose methotrexate.^{16, 17} We believe that agents that inhibit the upstream NLRP3 inflammasome and downstream activation of IL-6 will also be useful to address residual cardiovascular risk and are under consideration.

The SPIRE cardiovascular outcomes trials were stopped early due to high rates of development of neutralizing anti-drug antibodies.¹⁸ While bococizumab immunogenicity is associated with a less durable LDL reduction, treatment with bococizumab in the longer duration SPIRE-2 outcomes trial was nonetheless associated with a 21% (95% CI 3 to 35%; p=0.02) relative risk reduction in major cardiovascular events overall and a 14% (95% CI 2 to −25%) relative risk reduction per 1 mmol/1 LDL-C. These data are fully in line with benefits observed in the FOURIER trial.^{12, 19} Thus, it is believed that the findings presented hereinabove are unlikely to be explained by diminished bococizumab LDL-C lowering efficacy and likely to apply more broadly to this drug class. As in any post-hoc analysis, the findings presented hereinabove may be susceptible to residual confounding. In particular, subjects with persistent inflammatory risk were more likely to have cardiovascular risk factors and higher median on-treatment LDL-C. However, the multivariable analyses adjusted for achieved LDL-C levels and showed minimal, if any, attenuation in risk. Furthermore, as shown in CANTOS which enrolled on the basis of elevated hsCRP, this risk group is likely to benefit from anti-inflammatory therapy.

In sum, these contemporary randomized trial data demonstrate that elevated levels of on-treatment hsCRP remain a significant predictor of future vascular risk among atherosclerosis patients concomitantly treated with statins and PCSK9 inhibition. This evidence of residual inflammatory risk despite maximal LDL-C lowering suggests that a combination of inflammation inhibitors in addition to lipid lowering agents may offer additional opportunities for cardiovascular risk reduction at all cholesterol levels.

### REFERENCES

1. Ridker P M, Rifai N, Pfeffer M A, Sacks F M, Moye L A, Goldman S, Flaker G C and Braunwald E. Inflammation, pravastatin, and the risk of coronary events after myocardial infarction in patients with average cholesterol levels. Cholesterol and Recurrent Events (CARE) Investigators. Circulation. 1998; 98:839-44.
2. Ridker P M, Rifai N, Clearfield M, Downs J R, Weis S E, Miles J S, Gotto A M, Jr. and Air Force/Texas Coronary Atherosclerosis Prevention Study I. Measurement of C-reactive protein for the targeting of statin therapy in the primary prevention of acute coronary events. N Engl J Med. 2001; 344:1959-65.
3. Nissen S E, Tuzcu E M, Schoenhagen P, Crowe T, Sasiela W J, Tsai J, Orazem J, Magorien R D, O'Shaughnessy C, Ganz P and Reversal of Atherosclerosis with Aggressive Lipid Lowering I. Statin therapy, LDL cholesterol, C-reactive protein, and coronary artery disease. N Engl J Med. 2005; 352:29-38.
4. Ridker P M, Cannon C P, Morrow D, Rifai N, Rose L M, McCabe C H, Pfeffer M A, Braunwald E, Pravastatin or Atorvastatin E and Infection Therapy-Thrombolysis in Myocardial Infarction I. C-reactive protein levels and outcomes after statin therapy. N Engl J Med. 2005; 352:20-8.
5. Morrow D A, de Lemos J A, Sabatine M S, Wiviott S D, Blazing M A, Shui A, Rifai N, Califf R M and Braunwald E. Clinical relevance of C-reactive protein during follow-up of patients with acute coronary syndromes in the Aggrastat-to-Zocor Trial. Circulation. 2006; 114:281-8.
6. Ridker P M, Danielson E, Fonseca F A, Genest J, Gotto A M, Jr., Kastelein J J, Koenig W, Libby P, Lorenzatti A J, Macfadyen J G, Nordestgaard B G, Shepherd J, Willerson J T, Glynn R J and Group J T S. Reduction in C-reactive protein and LDL cholesterol and cardiovascular event rates after initiation of rosuvastatin: a prospective study of the JUPITER trial. Lancet. 2009; 373:1175-82.
7. Braunwald E. Creating controversy where none exists: the important role of C-reactive protein in the CARE, AFCAPS/TexCAPS, PROVE I T, REVERSAL, A to Z, JUPITER, HEART PROTECTION, and ASCOT trials. Eur Heart J. 2012; 33:430-2.
8. Ridker P M. Residual inflammatory risk: addressing the obverse side of the atherosclerosis prevention coin. Eur Heart J. 2016; 37:1720-2.
9. Ridker P M. How Common Is Residual Inflammatory Risk? Circ Res. 2017; 120:617-619.
10. Ridker P M, Everett B M, Thuren T, MacFadyen J G, Chang W H, Ballantyne C, Fonseca F, Nicolau J, Koenig W, Anker S D, Kastelein J J P, Cornel J H, Pais P, Pella D, Genest J, Cifkova R, Lorenzatti A, Forster T, Kobalava Z, Vida-Simiti L, Flather M, Shimokawa H, Ogawa H, Dellborg M, Rossi P R F, Troquay R P T, Libby P, Glynn R J and Group C T. Antiinflammatory Therapy with Canakinumab for Atherosclerotic Disease. N Engl J Med. 2017; 377:1119-1131.
11. Sabatine M S, Giugliano R P, Keech A C, Honarpour N, Wiviott S D, Murphy S A, Kuder J F, Wang H, Liu T, Wasserman S M, Sever P S, Pedersen T R, Committee F S and Investigators. Evolocumab and Clinical Outcomes in Patients with Cardiovascular Disease. N Engl J Med. 2017; 376:1713-1722.
12. Ridker P M, Revkin J, Amarenco P, Brunell R, Curto M, Civeira F, Flather M, Glynn R J, Gregoire J, Jukema J W, Karpov Y, Kastelein J J P, Koenig W, Lorenzatti A, Manga P, Masiukiewicz U, Miller M, Mosterd A, Murin J, Nicolau J C, Nissen S, Ponikowski P, Santos R D, Schwartz P F, Soran H, White H, Wright R S, Vrablik M, Yunis C, Shear C L, Tardif J C and Investigators SCO. Cardiovascular Efficacy and Safety of Bococizumab in High-Risk Patients. N Engl J Med. 2017; 376:1527-1539.
13. Ridker P M, Amarenco P, Brunell R, Glynn R J, Jukema J W, Kastelein J J, Koenig W, Nissen S, Revkin J, Santos R D, Schwartz P F, Yunis C, Tardif J C, Studies of P I and the Reduction of vascular Events I. Evaluating bococizumab, a monoclonal antibody to PCSK9, on lipid levels and clinical events in broad patient groups with and without prior cardiovascular events: Rationale and design of the Studies of PCSK9 Inhibition and the Reduction of vascular Events (SPIRE) Lipid Lowering and SPIRE Cardiovascular Outcomes Trials. Am Heart J. 2016; 178:135-44.
14. Ridker P M, Danielson E, Fonseca F A, Genest J, Gotto A M, Jr., Kastelein J J, Koenig W, Libby P, Lorenzatti A J, MacFadyen J G, Nordestgaard B G, Shepherd J, Willerson J T, Glynn R J and Group J S. Rosuvastatin to prevent vascular events in men and women with elevated C-reactive protein. N Engl J Med. 2008; 359:2195-207.
15. Tardif J C and L'Allier P. Colchicine Cardiovascular Outcomes Trial (COLCOT). www.clinicaltrials.gov/ct2/show/NCT02551094?term=colcat&ran=1. Accessed on Feb. 9, 2017.
16. Everett B M, Pradhan A D, Solomon D H, Paynter N, Macfadyen J, Zaharris E, Gupta M, Clearfield M, Libby P, Hasan A A, Glynn R J and Ridker P M. Rationale and design of the Cardiovascular Inflammation Reduction Trial: a test of the inflammatory hypothesis of atherothrombosis. Am Heart J. 2013; 166:199-207 e15.
17. Ridker P M, Tardif J C, Amarenco P, Duggan W, Glynn R J, Jukema J W, Kastelein J J P, Kim A M, Koenig W, Nissen S, Revkin J, Rose L M, Santos R D, Schwartz P F, Shear C L, Yunis C and Investigators S. Lipid-Reduction Variability and Antidrug-Antibody Formation with Bococizumab. N Engl J Med. 2017; 376:1517-1526.
18. Ference B A, Cannon C P, Landmesser U, Luscher T F, Catapano A L and Ray K K. Reduction of low density lipoprotein-cholesterol and cardiovascular events with proprotein convertase subtilisin-kexin type 9 (PCSK9) inhibitors and statins: an analysis of FOURIER, SPIRE, and the Cholesterol Treatment Trialists Collaboration. Eur Heart J. 2017.

What is claimed is:

1. A method of treating a cardiovascular disease, the method comprising administering to a subject in need thereof a therapeutically effective amount of a lipid lowering agent and an anti-inflammatory agent.

2. The method of claim 1, wherein the anti-inflammatory agent is a proinflammatory cytokine inhibitor.

3. The method of claim 1 or claim 2, wherein the anti-inflammatory agent comprises an IL-1 inhibitor, an IL-1 receptor (IL-1R) inhibitor, an IL-6 inhibitor, an IL-6 receptor (IL-6R) inhibitor, a NLRP3 inhibitor, a TNF inhibitor, an IL-8 inhibitor, an IL-18 inhibitor, an inhibitor of natural killer cells, or combinations thereof.

4. The method of any one of claims 1-3, wherein the anti-inflammatory agent is a nucleic acid, an aptamer, an antibody or antibody fragment, an inhibitory peptide, or a small molecule.

5. The method of claim 3 or claim 4, wherein the anti-inflammatory agent comprises an IL-1 inhibitor.

6. The method of claim 5, wherein the IL-1 inhibitor is an IL-1α inhibitor.

7. The method of claim 6, wherein the IL-1α inhibitor is an anti-sense oligonucleotide against IL-1α, MABp1, or sIL-1RI.

8. The method of claim 5, wherein the IL-1 inhibitor is an IL-1β inhibitor.

9. The method of claim 8, wherein the IL-1β inhibitor is an anti-sense oligonucleotide against IL-1β, canakinumab, gevokizumab, diacerein, LY2189102, CYT013, sIL-1RII, VX-740, or VX-765.

10. The method of claim 5, wherein the IL-1 inhibitor is suramin sodium, methotrexate-methyl-d3, methotrexate-methyl-d3 dimethyl ester, or diacerein.

11. The method of claim any one of claims 3-10, wherein the anti-inflammatory agent comprises an IL-1R inhibitor.

12. The method of claim 11, wherein the IL-1R inhibitor is an IL-1R antagonist.

13. The method of claim A11 or claim A12, wherein the IL-1R inhibitor is an anti-sense oligonucleotide against IL-1R, anakinra, Rilonacept, MEDI-8968, sIL-1RI, EBI-005, interleukin-1 receptor antagonist (IL-1RA), or AMG108.

14. The method of any one of claims 3-13, wherein the anti-inflammatory agent comprises an IL-6 inhibitor.

15. The method of claim 14, wherein the IL-6 inhibitor is an anti-sense oligonucleotide against IL-6, siltuximab, sirukumab, clazakizumab, olokizumab, elsilimomab, IG61, BE-8, CNTO328 PGE1 and its derivatives, PGI2 and its derivatives, or cyclophosphamide.

16. The method of any one of claims 3-15, wherein the anti-inflammatory agent comprises an IL-6R inhibitor.

17. The method of claim 16, wherein the IL-6R inhibitor is an IL-6R antagonist.

18. The method of claim 16 or claim 17, wherein the IL-6R inhibitor is an anti-sense oligonucleotide against IL-6R, tocilizumab, sarilumab, PM1, AUK12-20, AUK64-7, AUK146-15, MRA, or AB-227-NA.

19. The method of any one of claims 1-18, wherein the anti-inflammatory agent comprises a NLRP3 inhibitor.

20. The method of claim 19, wherein the NLPR3 inhibitors is an anti-sense oligonucleotide against NLPR3, colchicine, MCC950, CY-09, ketone metabolite beta-hydroxubutyrate (BHB), a type I interferon, resveratrol, arglabin, CB2R, Glybenclamide, Isoliquiritigenin, Z-VAD-FMK, or microRNA-223.

21. The method of any one of claims 3-20, wherein the anti-inflammatory agent comprises a TNF inhibitor.

22. The method of claim 21, wherein the TNF inhibitor is an anti-sense oligonucleotide against TNF, infliximab, adalimumab, certolizumab pegol, golimumab, etanercept (Enbrel), thalidomide, lenalidomide, pomalidomide, a xanthine derivative, bupropion, 5-HT2A agonist, or a hallucinogen.

23. The method of any one of claims 3-22, wherein the anti-inflammatory agent comprises an IL-8 inhibitor.

24. The method of claim 23, wherein the IL-8 inhibitor is an anti-sense oligonucleotide against IL8, HuMab-10F8, Reparixin, Curcumin, Antileukinate, Macrolide, or a trifluoroacetate salt.

25. The method of any one of claims 3-24, wherein the anti-inflammatory agent comprises an IL-18 inhibitor.

26. The method of claim 25, wherein the IL-18 inhibitor is an anti-sense oligonucleotide against IL-18, IL-18 binding protein, IL-18 antibody, NSC201631, NSC61610, or NSC80734.

27. The method of any one of claims 3-26, wherein the anti-inflammatory agent comprises an inhibitor of natural killer cells.

28. The method of claim 27, wherein the inhibitor of natural killer cells is an antibody targeting natural killer cells.

29. The method of any one of claims 1-28, wherein the anti-inflammatory agent comprises methotrexate.

30. The method of any one of claims 1-29, wherein the anti-inflammatory agent comprises arhalofenate.

31. The method of any one of claims 1-30, wherein the lipid lowering agent comprises a proprotein convertase subtilisin/kexin type 9 (PCSK9) inhibitor.

32. The method of any one of claims 1-30, wherein the PCSK9 inhibitor is a natural PCSK9 inhibitor, a PCSK9 antibody, an antisense nucleic acid, a peptide inhibitor, a PCSK9 vaccine, or a small molecule inhibitor.

33. The method of claim 32, wherein the natural PCSK9 inhibitor is berberine, annexin A2, or adnectin.

34. The method of claim 32, wherein the small molecule inhibitor is PF-06446846, anacetrapib, or K-312.

35. The method of claim 32, wherein the PCSK9 antibody is alirocumab, evolocumab, 1D05-IgG2, RG-7652, LY3015014, or bococizumab.

36. The method of claim 32, wherein the antisense nucleic acid is an RNAi molecule.

37. The method of claim 36, wherein the RNAi molecule is inclisiran or ALN-PCS.

38. The method of claim 32, wherein the peptide inhibitor is a peptide that mimics an EGFa domain of low-density lipoprotein receptor (LDL-R).

39. The method of claim 32, wherein the PCSK9 vaccine comprises an antigenic PCSK9 peptide.

40. The method of any one of claims 1-39, wherein the lipid lowering agent comprises a HMG-CoA reductase inhibitor.

41. The method of claim 40, wherein the HMG-CoA reductase inhibitor is a statin.

42. The method of claim 41, wherein the statin is simvastatin, lovastatin, pravastatin, fluvastatin, atorvastatin, cerivastatin, rosuvastatin, or pitivastatin.

43. The method of any one of claims 1-42, wherein the lipid lowering agent is a fibric acid derivative (fibrate), a bile acid sequestrant, a resin, a nicotinic acid agent, a cholesterol absorption inhibitor, acyl-coenzyme A, a cholesterol acyl transferase (ACAT) inhibitor, a cholesteryl ester transfer protein (CETP) inhibitor, a LDL receptor antagonist, a farnesoid X receptor (FXR) antagonist, a sterol regulatory binding protein cleavage activating protein (SCAP) activator, a microsomal triglyceride transfer protein (MTP) inhibitor, a squalene synthase inhibitor, or a peroxisome proliferation activated receptor (PPAR) agonist.

44. The method of any one of claims 1-43, wherein the lipid lowering agent and the anti-inflammatory agent are administered together.

45. The method of any one of claims 1-43, wherein the lipid lowering agent and the anti-inflammatory agent are administered separately.

46. The method of any one of claims 1-45, wherein the lipid lowering agent and/or the anti-inflammatory agent is administered intranasally, intravenously, intramuscularly, subcutaneously, or orally.

47. The method of any one claims 1-46, wherein the level or activity of a proinflammatory cytokine in the subject is reduced.

48. The method of any one of claims 1-47, wherein the level or activity of C-reactive protein (CRP) in the subject is reduced.

49. The method of any one of claims 1-48, wherein the level or activity of non-high-density lipoprotein (HDL)-cholesterol in the subject is reduced.

50. The method of any one of claims 1-49, wherein the level or activity of LDL-cholesterol in the subject is reduced.

51. The method of any one of claims 1-50, wherein the level or activity of total cholesterol in the subject is reduced.

52. The method of any one of claims 1-51, wherein the level or activity of apolipoprotein B (ApoB) in the subject is reduced.

53. The method of any one of claims 1-52, wherein the level or activity of triglycerides in the subject is reduced.

54. The method of any one of claims 1-53, wherein the ratio of total cholesterol to HDL-cholesterol in the subject is reduced.

55. The method of any one of claims 1-54, wherein the occurrence of non-fatal myocardial infarction is reduced.

56. The method of any one of claims 1-54, wherein the occurrence of non-fatal stroke is reduced.

57. The method of any one of claims 1-54, wherein the rate of cardiovascular mortality is reduced.

58. The method of any one of claims 1-57, wherein the cardiovascular disease is myocardial infarction, stroke, acute coronary syndrome, myocardial ischemia, chronic stable angina pectoris, unstable angina pectoris, cardiovascular death, coronary re-stenosis, coronary stent re-stenosis, coronary stent re-thrombosis, recurrent cardiovascular events, revascularization, angioplasty, transient ischemic attack, pulmonary embolism, vascular occlusion, or venous thrombosis.

59. A method of reducing a recurrence rate of a cardiovascular disease in a subject who has received or is undergoing therapy with a lipid lowering agent, the method comprising administering to the subject an effective amount of an anti-inflammatory agent.

60. A method of predicting a recurrence rate of a cardiovascular disease in a subject who has received or is undergoing therapy with the lipid lowering agent, the method comprising measuring a level of C-reactive protein (CRP) in the subject and determining that the subject is likely to have recurrence of the cardiovascular disease if the CRP level is above a pre-determined value.

61. The method of claim 60, wherein the pre-determined value is 3 mg/L.

62. The method of claim 60, wherein the pre-determined value is 2 mg/L.

63. The method of claim 60, wherein the pre-determined value is 1 mg/L.

64. A method of treating a cardiovascular disease, the method comprising administering to a subject in need thereof a therapeutically effective amount of a bispecific antibody comprising a first antigen-binding domain that binds a proinflammatory cytokine and a second antigen-binding domain that binds a proprotein convertase subtilisin/kexin type 9 (PCSK9).

65. The method of claim 64, wherein the proinflammatory cytokine is IL-1, IL-1 receptor (IL-1R), IL-6, IL-6 receptor (IL-6R), NLRP3, TNF, IL-8, or IL-18.

66. The method of claim 65, wherein the first antigen-binding domain binds to IL-1.

67. The method of claim 66, wherein the first antigen-binding domain binds to IL-1α.

68. The method of claim 66, wherein the first antigen-binding domain is derived from MABp1.

69. The method of claim 66, wherein the first antigen-binding domain binds to IL-1β.

70. The method of claim 69, wherein the first antigen-binding domain is derived from canakinumab, diacerein, gevokizumab, or LY2189102.

71. The method of claim 65, wherein the first antigen-binding domain binds to IL-1R.

72. The method of claim 71, wherein the first antigen-binding domain is derived from MEDI-8968 or AMG108.

73. The method of claim 65, wherein the first antigen-binding domain binds to IL-6.

74. The method of claim 73, wherein the first antigen-binding domain is derived from siltuximab, sirukumab, clazakizumab, olokizumab, or elsilimomab.

75. The method of claim 65, wherein the first antigen-binding domain binds to IL-6R.

76. The method of claim 75, wherein the first antigen-binding domain is derived from tocilizumab, sarilumab, PM1, AUK12-20, AUK64-7, AUK146-15, or AB-227-NA.

77. The method of claim 65, wherein the first antigen-binding domain binds to NLRP3.

78. The method of claim 77, wherein the first antigen-binding domain is derived from an anti-NLRP3 antibody.

79. The method of claim 65, wherein the first antigen-binding domain binds to TNF.

80. The method of claim 79, wherein the first antigen-binding domain is derived from infliximab, adalimumab, certolizumab pegol, golimumab, or etanercept (Enbrel).

81. The method of claim 65, wherein the first antigen-binding domain binds to IL-8.

82. The method of claim 81, wherein the first antigen-binding domain is derived from HuMab-10F8.

83. The method of claim 65, wherein the first antigen-binding domain binds to IL-18.

84. The method of claim 83, wherein the first antigen-binding domain is derived from an IL-18 antibody.

85. The method of any one of claims 64-84, wherein the second antigen-binding domain is derived from alirocumab, evolocumab, 1D05-IgG2, RG-7652, LY3015014, or bococizumab.

86. The method of any one of claims 64-85, wherein the bispecific antibody comprises a common Fc region.

87. The method of any one of claims 64-86, wherein the bispecific antibody is a monoclonal bispecific antibody.

88. The method of any one of claims 64-87, further comprising administering to the subject a therapeutically effective amount of a HMG-CoA reductase inhibitor.

89. The method of claim 88, wherein the HMG-CoA reductase inhibitor is a statin.

90. The method of claim 89, wherein the statin is simvastatin, lovastatin, pravastatin, fluvastatin, atorvastatin, cerivastatin, rosuvastatin, or pitivastatin.

91. The method of any one of claims 64-89, wherein the bispecific antibody is administered intravenously, intramuscularly, subcutaneously, or orally.

92. The method of any one claims 64-91, wherein the level or activity of a proinflammatory cytokine in the subject is reduced.

93. The method of any one of claims 64-92, wherein the level or activity of C-reactive protein (CRP) in the subject is reduced.

94. The method of any one of claims 64-93, wherein the level or activity of non-high-density lipoprotein (HDL)-cholesterol in the subject is reduced.

95. The method of any one of claims 64-94, wherein the level or activity of LDL-cholesterol in the subject is reduced.

96. The method of any one of claims 64-95, wherein the level or activity of total cholesterol in the subject is reduced.

97. The method of any one of claims 64-96, wherein the level or activity of apolipoprotein B (ApoB) in the subject is reduced.

98. The method of any one of claims 64-97, wherein the level or activity of triglycerides in the subject is reduced.

99. The method of any one of claims 64-98, wherein the ratio of total cholesterol to HDL-cholesterol in the subject is reduced.

100. The method of any one of claims 64-99, wherein the occurrence of non-fatal myocardial infarction is reduced.

101. The method of any one of claims 64-99, wherein the occurrence of non-fatal stroke is reduced.

102. The method of any one of claims 64-101, wherein the rate of cardiovascular mortality is reduced.

103. A method of treating a cardiovascular disease, the method comprising administering to a subject in need thereof a therapeutically effective amount of a bispecific antibody comprising a first antigen-binding domain that binds IL-1 and a second antigen-binding domain that binds a proprotein convertase subtilisin/kexin type 9 (PCSK9).

104. The method of claim 103, wherein the first antigen-binding domain binds to IL-1α.

105. The method of claim 104, wherein the first antigen-binding domain is derived from MABp1.

106. The method of claim 103, wherein the first antigen-binding domain binds to IL-1β.

107. The method of claim 106, wherein the first antigen-binding domain is derived from canakinumab, diacerein, gevokizumab, or LY2189102.

108. The method of any one of claims 103-107, wherein the second antigen-binding domain is derived from alirocumab, evolocumab, 1D05-IgG2, RG-7652, LY3015014, or bococizumab.
